(19) United States
(12) Patent Application Publication
(10) Pub. No.: US 2006/0248601 A1
(43) Pub. Date: Nov. 2, 2006

(54) Modification of organelle metabolism by unc-51-like kinases roma1 or 2tm proteins

(76) Inventors: Arnd Steuernagel, Gottingen (DE); Bronner Gunter, Gottingen (DE); Ciossek Thomas, Gottingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/473,404

(22) PCT Filed: Mar. 27, 2002
(86) PCT No.: PCT/EP02/03465

§ 371 Date: Apr. 23, 2004

(30) Foreign Application Priority Data
Mar. 30, 2001 (EP) 011082153
Jul. 16, 2001 (EP) 011172285
Jul. 23, 2001 (EP) 011178704

(51) Int. Cl. A01K 67/027; A01K 67/033; A61K 48/00; C12N 9/12; A61K 38/48

(52) U.S. Cl. 800/8; 424/94.5; 514/44; 435/194; 435/325; 435/320.1

(57) ABSTRACT

The invention discloses polypeptides (Unc-51 kinase, ROMA1, and/or 2TM protein) affecting the activity of Uncoupling Proteins (UCPs), thereby leading to an altered mitochondrial activity and thus contributing to membrane stability and/or function of organelles, preferably mitochondria. This invention relates to the use of these polypepetides in the diagnosis, study, prevention, and treatment of diseases and disorders, for example, but not limited to, metabolic diseases such as obesity, adipositas, eating disorders, wasting syndromes (cachexia), pancreatic dysfunctions (for example diabetes), disorders related to ROS production, and others.

This invention relates to nucleic acid and amino acid sequences of proteins referred to herein as Unc-51-like kinases, Regulators Of Mitochondrial Activity (ROMA1), and mitochondrial 2TM proteins. Further, this invention relates to mutations in Unc-51, ROMA1, and/or 2TM proteins that affect the activity of uncoupling proteins (UCPs), thereby leading to an altered mitochondrial activity. This invention relates also to the use of these sequences in the diagnosis, study, prevention, and treatment of diseases and disorders, for example, but not limited to, metabolic diseases such as obesity, adipositas, eating disorders, wasting syndromes (cachexia), pancreatic dysfunctions (for example diabetes), mitochondrial disorders, disorders related to ROS production, and others.

Mitochondria are the energy suppliers of animal cells. Most of the energy available from metabolising foodstuffs like carbohydrates, fats etc. is used to create a proton gradient across the inner mitochondrial membrane. This proton gradient drives the enzyme ATP synthetase that produces ATP, the cells major fuel substance (Mitchell P, Science 206, 1979, 1148-1159). In the mitochondria of brown adipose tissue (BAT) exists a protein (Uncoupling Protein 1, UCP1) that tunnels protons through the inner mitochondrial membrane (review in Klingenberg et al., 1999, Biochim. Biophys. Acta, 1415(2):271-96). The energy stored in the proton gradient is thereby released as heat and not used for ATP synthesis.

When the energy intake of an animal exceeds expenditure surplus energy can be stored as fat in adipose tissue. The generation of a proton leak across the inner mitochondrial membrane by the activation of uncoupling proteins would reduce caloric efficiency and thus avoid the accumulation of excess body fat (obesity) that is detrimental to the animals health. In human, however, brown adipose tissue is almost absent in adults. Therefore, UCP1 was not considered to be a major factor in the formation or prevention of human obesity. Recently the discovery of further proteins of similar sequence (UCP2-UCP5) that are widely expressed in human tissues (e.g. white adipose tissue, muscle) made this members of the UCP family to important targets for pharmaceutical research (reviewed in Adams 2000, Nutr., 130(4):711-4). Interestingly, and as reviewed in Ricquier, 2000, Biochem J. 345, 161-179, further homologues have been identified, like, inter alia, the plant UCPs StUCP (from Solanum tuberculosum) and AtUCP (Arabidopsis thaliana). Although the in vivo function of these proteins is still unknown, the possibility to influence UCP activity would be a conceivable therapy for the treatment or prevention of obesity and related diseases.

Mitochondria have a very specialized function in energy conversion and said function is reflected in their morphological structure, namely the distinct internal membrane. This internal membrane does not only provide the framework for electron-transport processes but also creates a large internal compartment in each organelle in which highly specialized enzymes are confined. Therefore, there is a strong relationship between mitochondrial energy metabolism and the biochemical/biophysical properties of these organelles.

The technical problem underlying the invention was to provide for means and methods for modulating the biological/biochemical activities of mitochondria and, thereby, modulating metabolic conditions in eukaryotic cells which influence energy expenditure, body temperature, thermogenesis, cellular metabolism to an excessive or deficient supply of substrate(s) in order to regulate the ATP level, the NAD⁺/NADH ratio, and/or superoxide production. The solution to this technical problem is achieved by providing the embodiments characterized in the claims.

As shown in the appended examples, this invention discloses genes that can suppress or enhance the eye defect induced by the activity of dUCPy. These genes are the Drosophila homologues of Unc-51 (enhancer), ROMA1 (regulator of mitochondrial activity) (suppressor) and mitochondrial 2TM protein (suppressor). It is envisaged that mutations in Unc-51, ROMA1, and/or 2TM in eukaryotic organisms affect the activity of Uncoupling proteins (UCPs) thereby leading to an altered mitochondrial activity.

Unc-51 was originally discovered in C. elegans as a gene required for axonal elongation and guidance (Ogura et al., 1994, Genes Dev 8:2389-2499; Ogura et al., 1997, Genes Dev 11: 1801-1811). A mouse homologue of Unc51 (called Ulk-1 for Unc-51 kinase 1) has been identified based on sequence homology to the C. elegans Unc-51 gene (Yan et al., 1998, Biochem Biophys Res Commun 246: 222-227). Later a second murine Unc-51-like kinase has been discovered called Ulk-2 (Yan et al., 1999, Oncogene 18:5850-5859). Human Ulk-1 was cloned based on sequence homology as well (Kuroyanagi et al., 1998, Genomics 51:76-85). The human gene is expressed ubiquitously, whereas the C. elegans gene is specifically expressed in neurons. A human Ulk-2 gene has not been reported in the literature. However, its existence can be deduced from genebank database entries. Sequence characteristics suggest that Unc-51-like genes form a subfamily of protein kinases, which are structurally conserved among eukaryotes (Yan et al., 1998, Biochem Biophys Res. Commun 246: 222-227). With exception of the C. elegans gene no function is known for Unc-51 like genes in higher eukaryotes.

Prohibitins are ubiquitous, abundant and evolutionary strongly conserved proteins that play a role in cellular processing, including cell cycle regulation, apoptosis, assembly of the mitochondrial respiratory chain enzymes, and agin (Coates et al., Exp Cell Res. 2001, 265:262-73). The mouse homolog BAP37 (synonyms are Phb2p, prohibitin 2) of Drosophila ROMA1 has been identified as an interactor of the IgM antigen receptor (Terashima et al. EMBO J. 1994 Aug. 15; 13(16):3782-92); human BAP-37 (synonym REA) has been identified as an interactor of the estrogen receptor (Montano et al, 1999. Proc Natl Acad Sci USA. 96(12):6947-52). BAP37 (prohibitin 1; REA) and prohibitin (prohibitin 1) interact with each other to form a complex in the inner mitochondrial membrane (see, Coates et a.l 1997, Curr. Biol.; 7(8):607-10). The yeast homologs Phb1p and Phb2p act as chaperones in the inner mitochondrial membrane that stabilize mitochondrial translation products (Nijtmans et al. 2000, EMBO J.; 19(11):2444-51).

The 2TM gene of Drosophila has conserved homologues in vertebrates (see FIG. 7). Sequence analysis of Drosophila, mouse, and human 2TM genes as shown in this invention predicts that the proteins encoded by the 2TM genes have two transmembrane domains (see FIG. 8).

Even if several candidate genes have been described which are supposed to influence the homeostatic system(s) that regulate body mass/weight, like leptin, VCPI, VCPL, or the peroxisome proliferator-activated receptor-gamma co-activator, the distinct molecular mechanisms and/or molecules influencing obesity or body weight/body mass regulations are not known.

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates to genes with novel functions in body-weight regulation, energy homeostasis, metabolism, and obesity. The present invention discloses a specific gene involved in the regulation of body-weight, energy homeostasis, metabolism, and obesity, and thus in disorders related thereto such as eating disorder, cachexia, diabetes mellitus, hypertension, coronary heart disease, hypercholesterolemia, dyslipidemia, osteoarthritis, gallstones, cancer, e.g. cancers of the reproductive organs, sleep apnea, disorders involved in ROS production, mitochondrial disorders, and neurodegenerative disorders.

More particularly, the present invention describes the human Unc51-kinases, ROMA1, and/or mitochondrial 2TM genes as being involved in those conditions mentioned above. So far, it has not been described that Unc51-kinases, ROMA1, and/or mitochondrial 2TM and homologous human proteins are involved in the regulation of energy homeostasis and body-weight regulation and related disorders, and thus, no functions in metabolic diseases and other diseases as listed above have been discussed. In this invention we demonstrate that the correct gene dose of Unc51-kinases, ROMA1, and/or mitochondrial 2TM is essential for maintenance of energy homeostasis. A genetic screen was used to identify that mutation of a Unc51-kinases, ROMA1, and/or mitochondrial 2TM homologous gene causes obesity.

Futher, this invention relates to proteins referred to as Unc51-like kinases, ROMA1, and/or mitochondrial 2TM proteins contributing to membrane stability and/or function of organelles, in particular mitochondria. This invention also relates to mutations in Unc-51, ROMA1, and/or 2TM that affect the activity of uncoupling proteins (UCPs), thereby leading to an altered mitochondrial activity. This invention also relates to the use of these sequences in the diagnosis, study, prevention, and treatment of diseases and disorders, for example, but not limited to, metabolic diseases such as obesity, adipositas, eating disorders, wasting syndromes (cachexia), mitochondrial disorders, pancreatic dysfunctions (for example diabetes), disorders related to ROS production, neurodegenerative disorders, and others.

The present invention relates to a nucleic acid molecule encoding a polypeptide contributing to membrane stability and/or function of organelles, in particular mitochondria, wherein said nucleic acid molecule (a) hybridizes under herein defined conditions to the complementary strand of a nucleic acid molecule encoding the amino acid sequence disclosed herein; (b) hybridizes under herein defined conditions to the complementary strand of a nucleic acid molecule as disclosed herein; (c) it is degenerate with respect to the nucleic acid molecule of (a); (d) encodes a polypeptide which is at least 85%, preferably at least 90%, more preferably at least 95%, more preferably at least 98% and up to 99.6% identical to a herein disclosed amino acid sequence representing a polynucleotide contributing to membrane stability and/or function of organelles; (e) differs from the nucleic acid molecule of (a) to (e) by mutation and wherein said mutation causes an alteration, deletion, duplication or premature stop in the encoded polypeptide or (f) has a sequence as depicted herein. Furthermore, the invention provides for vectors comprising said nucleic acid molecule as well as to hosts transformed with said vector. The invention also relates to polypeptides encoded by said nucleic acid molecules and to antibodies, fragments or derivatives thereof or an aptamer or another receptor specifically recognizing the nucleic acid molecule or the polypeptide of the invention. The invention also describes compositions comprising nucleic acid molecules, vectors, hosts, polypeptides, fusion proteins, antibodies, fragment or derivative thereof or aptamers or other receptors or anti-sense oligonucleotides of the invention. Preferably these compositions are diagnostic compositions or pharmaceutical compositions. Furthermore, the invention provides for methods of identifying a polypeptide or (a) substance(s) involved in cellular metabolism in an animal or an plant or capable of modifying homeostasis and for identifying a polypeptide involved in the regulation of body weight in a mammal. The invention also relates to methods of identifying a compound influencing the expression of the nucleic acid molecule or the polypeptide of the invention. In addition, methods are disclosed for assessing the impact of the expression of one or more compounds of the invention. Finally, the invention provides for compositions comprising inhibitors and/or stimulators of the (poly)peptide of the invention and it provides for kits comprising the compounds of the invention.

This invention is based on the identification of a protein (referred to as Unc-51, ROMA1, and/or 2TM) contributing to membrane stability and/or function of organelles, preferrably mitochondria. It was found by the inventors that mutations in Unc-51, ROMA1, and/or 2TM affect the activity of Uncoupling Proteins (UCPs), thereby leading to an altered mitochondrial activity. Thus, these sequences may be used in the diagnosis, study, prevention, and treatment of diseases and disorders, for example, but not limited to, metabolic diseases such as obesity, adipositas, eating disorders, wasting syndromes (cachexia), pancreatic dysfunctions (for example diabetes), mitochondrial disorders, hypercholesterolemia, dyslipidemia, coronary heart disease, osteoarthritis, gallstones, cancers of the reproductive organs, sleep apnea, disorders related to ROS production, and others.

Accordingly, the present invention relates to a nucleic acid molecule encoding a polypeptide contributing to membrane stability and/or function of organelles, for example, mitochondria, wherein said nucleic acid molecule
(a) hybridizes at 65° C. or 66° C. in a solution containing 0.2×SSC and 0.1% SDS to the complementary strand of a nucleic acid molecule encoding the amino acid sequence of the protein described in this invention;
(b) it is degenerate with respect to the nucleic acid molecule of (a);
(c) encodes a polypeptide which is at least 35%, preferably at least 50%, more preferably at least 60%, more preferably at least 70%, more preferably at least 80%, more preferably at least 90%, most preferably at least 95% and most preferably at least 99% identical to the amino acid sequence of the protein of the invention;
(d) differs from the nucleic acid molecule of (a) to (c) by mutation and wherein said mutation causes an alteration, deletion, duplication or premature stop in the encoded polypeptide.

As documented in the appended examples, the present invention provides for genes and gene products which are either directly or indirectly involved in membrane stability and/or function of organelles, in particular of mitochondria.

The term “membrane stability” as used herein comprises not only the overall stability but also comprises local stabilities on membranes of organelles, for example of the inner and outer membrane, but in particular of the inner membrane. The term “membrane stability” relates, therefore, to structural features of the membranes, provided by protein-protein interactions as well as by protein-lipid interactions leading to a defined membrane composition.

The term “contributing to membrane function of organelles” as employed herein above relates to functions of the above defined polypeptide comprising, inter alia, transport functions (like active and passive transport of ions, metabolites, vitamines, etc.), regulator functions of other membrane proteins (like transporters, carriers) or modifier functions of other (membrane) proteins (like enhancement/suppressor functions) and/or other functions as defined herein below.

The term “organelles” as employed herein not only relates to mitochondria but also to further organelles, for example, but not limited to, peroxisomes or plant cell organelles, e.g. chloroplasts.

The terms “hybridizes” and “hybridizing” as employed in context of the present invention preferably relate to stringent conditions as, inter alia, defined herein above, e.g. 0.2×SSC, 0.1% SDS at 65° C. or 66° C. Said conditions comprise hybridization as well as washing conditions. However, it is preferred that washing conditions are more stringent than hybridization conditions. By setting the conditions for hybridization, the person skilled in the art can determine if strictly complementary sequences or sequences with a higher or lower degree of homology are to be detected. The setting of conditions is well within the skill of the artisan and to be determined according to protocols described, for example, in Sambrook, Molecular Cloning, A Laboratory Manual, 2^{nd} edition (1989), Cold Spring Harbor Laboratory Press, Cold Spring Harbor, N.Y. Non-stringent hybridization conditions for the detection of homologous and not exactly complementary sequences may be set at 6×SSC, 1% SDS at 65° C. or 66° C.

The molecules hybridizing to the nucleic acid molecules of the invention also comprise fragments, derivatives and allelic variants of the above-described nucleic acid molecules which encode (poly)peptides regulating, causing or contributing to obesity described in the present invention. In this regard, fragments are defined as parts of the nucleic acid molecules, which are long enough in order to encode said (poly)peptides. The term derivatives means that the sequences of these hybridizing molecules differ from the sequences of the above-mentioned nucleic acid molecules at one or more positions and that they exhibit a high degree of homology to these sequences. The person skilled in the art may employ computer programs and packages in order to determine homology values. Generally, nucleotide or amino acid sequence identities/homologies can be determined conventionally by using known computer programs such as BLASTIN, BLASTP, NALIGN, PALIGN, or bl2seq using particular algorithms to find the best segment of homology between two segments.

As shown in the appended examples, in the context of the present invention the comparative analysis of the percentage of identities at the amino acid level are preferably obtained using the “bl2seq” program from NCBI using the following parameters: Open Gap Cost: 11 and Gap Extension Cost: 1. However, the program allows any positive integer for said value(s).

Homology means that functional and/or structural equivalence exists between the respective nucleic acid molecules or the proteins they encode. The nucleic acid molecules, which are homologous to the above-described molecules and represent derivatives of these molecules, are generally variations of these molecules that constitute modifications which exert the same biological function. These variations may be naturally occurring variations, for example sequences derived from other organisms, or mutations, whereby these mutations may have occurred naturally or they may have been introduced by means of a specific mutagenesis. Moreover, the variations may be synthetically produced sequences. The allelic variants may be naturally occurring as well as synthetically produced variants or variants produced by recombinant DNA techniques.

It is preferred that the nucleic acid molecule of the invention encodes a polypeptide contributing to membrane stability and/or function of organelles which is at least 85% and up to 99.6% identical to the amino acid sequence of the protein of the invention and represents a protein which has surprisingly been found to be involved in membrane function and/or stability of organelles and has, in particular, be found to be able to modify UCPs; see also appended examples. As demonstrated in the appended examples, the here described polypeptide (and encoding nucleic acid molecule) was able to modify, e.g. suppress or enhance a specific eye phenotype in Drosophila which was due to the overexpression of the Drosophila melanogaster gene dUCPy. The overexpression of dUCP (with homology to human UCPs) in the compound eye of Drosophila led to a clearly visible eye defect (see appended Examples and figures) which can be used as a “read-out” for a genetical “modifier screen”.

In said “modifier screen” thousands of different genes are mutagenized to modify their expression in the eye. Should one of the mutagenized genes interact with dUCPy and modify its activity an enhancement or suppression of the eye defect will occur. Since such flies are easily to discern they can be selected to isolate the interacting gene.

As shown in the appended examples, several genes were identified that can suppress or enhance the eye defect induced by the activity of dUCPy. The sequences of the Drosophila genes were used to perform a BLAST search for mammalian homologues in public databases (e.g., National Center for Biotechnology Information (NCBI) at the National Institutes of Health (NIH)).

The sequence similarities between the Unc-51-like genes from Drosophila, mouse, and human are shown in Table 1 (the alignment of the protein sequences is shown in FIG. 2):

TABLE 1
Unc-51-like genes and proteins of the invention
Table 1A: Genbank (NCBI) accession numbers of Unc-51-like genes
and proteins of the invention

| | | Accession | |
|---|---|---|---|
| | | Number | Accession Number |
| Species | Name | (protein) | (cDNA) |
| Drosophila | | AAF49878 | CG10967 |
| melanogaster | | (GenBank) | (Drosophila genome |
| | | | project, Berkeley) |
| Mouse | Unc-51 like kinase 1 | NP_033495 | NM_009469 |
| | (ULK-1) | (GenBank) | (GenBank) |
| Mouse | Unc-51-like kinase | BAA77341 | AB019577 |
| | (ULK-2) | (GenBank) | (GenBank) |
| Human | Unc-51 like kinase 1 | NP_003556 | NM_003565 |
| | (ULK-1) | (GenBank) | (GenBank) |
| Human | KIAA0623 gene | NP_055498 | NM_014683 |
| | product (ULK-2) | (GenBank) | (GenBank) |

Table 1B: Similarities and identities between Unc-51-like proteins

| | | | | along X amino |
|---|---|---|---|---|
| Protein 1 | Protein 2 | identity | similarity | acids |
| Drosophila | mouse ULK 1 | 43% | 53% | 611 |
| Unc-51 | | | | |
| Drosophila | mouse ULK 2 | 39% | 51% | 616 |
| Unc-51 | | | | |
| Drosophila | human ULK 1 | 32% | 42% | 1062 |
| Unc-51 | | | | |
| Drosophila | human | 32% | 46% | 1047 |
| Unc-51 | KIAA0623 | | | |
| mouse ULK 1 | human ULK 1 | 89% | 91% | 1054 |
| mouse ULK 1 | human | 52% | 63% | 1072 |
| | KIAA0623 | | | |
| mouse ULK 2 | human ULK 1 | 51% | 63% | 1080 |
| mouse ULK 2 | human | 93% | 95% | 1037 |
| | KIAA0623 | | | |
| human ULK 1 | human | 52% | 63% | 1083 |
| | KIAA0623 | | | |

Another modifying gene is called Regulator Of Mitochondrial Activity 1 (ROMA1). The sequence of the Drosophila gene was used to perform a BLAST search for mammalian homologues in public databases (National Center for Biotechnology Information (NCBI) at the National Institutes of Health (NIH)). The human homologue of ROMA1 is annotated as “B-cell associated protein” (BAP) under GenBank accession number XP_{—}006639.1 (Identities=189/261 (72%), Positives=236/261 (90%); see also Accession Number AX146891 for BAP1, disclosed in patent application WO 01/36674), the mouse homologue is available under accession number NP_{—}031557.1 (Identities=185/260 (71%), Positives=231/260 (88%)) (see FIG. 5).

Another modifying gene is called 2TM. The sequence similarities between the 2TM genes from Drosophila, mouse and human are shown in the following Table 2 (alignment of the protein sequences is shown in FIG. 7):

Table 2. 2TM Genes and Proteins of Different Species

Table 2 A: Genbank (NCBI) Accession Numbers of 2TM Genes and Proteins of the Invention

Drosophila melanogaster: GadFly Accession Number CG7620 (Drosophila genome project, Berkeley), NCBI Accession Number AAF454894

Mouse: GenBank Accession Number BAB26124

Human (see also FIG. 7):

Chromosome 1 protein (GenBank Accession Number XP_{—}057659; SEQ ID NO: 6; FIG. 7A). A cDNA from his locus (Accession No. AX026549) is mentioned in WO 00/40752. This document relates to “Cancer associated genes and their products”.

HYPERLINK Chromosome 10 protein EnsEMBL human genome annotation Accession Number ENSP00000242518 gene:ENSG00000122914 genomic clone:AL360177 (see SEQ ID NO: 7, FIG. 7B)

Chromosome 2 protein EnsEMBL human genome annotation Accession Number ENSP00000243785, gene:ENSG00000123998, genomic clone:AC044850 (see SEQ ID NO: 8, FIG. 7C)

Chromosome 17 protein EnsEMBL human genome annotation Accession Number ENSP00000250594 gene:ENSG00000129653 genomic clone:AC015802 (not complete) (see SEQ ID NO: 9, FIG. 7D)

TABLE 2B
Similarities and identities between 2TM proteins from
Drosophila melanogaster (GadFly accession number CG7620), mouse
(GenBank accession number BAB26124) and human SEQ ID NO: 6,
SEQ ID NO: 7, SEQ ID NO: 8, SEQ ID NO: 9

| | | | | Along XX amino |
|---|---|---|---|---|
| Acc. No. | Acc. No. | Identity | Similarity | acids |
| CG7620 | SEQ ID NO: 6 | 42% | 56% | 97 |
| CG7620 | SEQ ID NO: 7 | 42% | 56% | 97 |
| CG7620 | SEQ ID NO: 8 | 40% | 52% | 101 |
| CG7620 | SEQ ID NO: 9 | 33% | 51% | 54 |
| SEQ ID NO: 6 | SEQ ID NO: 8 | 80% | 83% | 118 |
| SEQ ID NO: 6 | SEQ ID NO: 9 | 82% | 85% | 111 |
| SEQ ID NO: 7 | SEQ ID NO: 9 | 86% | 91% | 58 |
| SEQ ID NO: 8 | SEQ ID NO: 9 | 85% | 89% | 64 |
| CG7620 | mouse | 39% | 52% | 110 |
| | BAB26124 | | | |

It is envisaged that mutations in the herein described polypeptides (and genes) lead to phenotypic and/or physiological chances which may comprise a modified and altered mitochondrial activity. This, in turn, may lead to, inter alia, an altered energy metabolism, altered thermogenesis and/or altered energy homeostasis.

In a preferred embodiment the above described nucleic acid molecule of the invention is DNA. In this context, it is understood that the term “nucleic acid molecule” comprises coding and, wherever applicable, non-coding sequences, like, inter alia, 5′ and 3′ non-coding sequences. Said 5′ and/or 3′ non-coding regions may comprise (specific) regulatory sequences ensuring initiation of transcription and optionally poly-A signals ensuring termination of transcription and/or stabilization of the transcript. Additional 5′ and 3′ non-coding regions may comprise promoters and/or transcriptional as well as translational enhancers. Furthermore, the term “nucleic acid molecule” may comprise intron(s) and splice variants, where applicable. The nucleic acid molecule may be a single-stranded or double stranded molecule, e.g. a DNA or an RNA. The term DNA as used herein comprises, inter alia, cDNA as well as genomic DNA. Furthermore, the nucleic acid molecule of the invention may also be an RNA molecule such as mRNA. In accordance with the present invention, the term “nucleic acid molecule” comprises also any feasible derivative of a nucleic acid to which a nucleic acid probe may hybridize. Said nucleic acid probe itself may be a derivative of a nucleic acid molecule capable of hybridizing to said nucleic acid molecule or said derivative thereof. The term “nucleic acid molecule” further comprises peptide nucleic acids (PNAs) containing DNA analogs with amide backbone linkages (Nielson, Science 254 (1991), 1497-1500).

In this context it has to be stressed that nucleic acid molecules of the invention may also be chemically synthesized, using, inter alia, synthesizers which are known in the art and commercially available, like, e.g. the ABI 394 DNA-RAN-synthesizers.

It is preferred that the nucleic acid molecule of the invention encodes a polypeptide contributing to membrane stability and/or function of organelles, wherein said polypeptide contributing to membrane stability and/or function in organelles is expressed in mitochondria and/or peroxisomes. It is particularly preferred that said polypeptide participates in the maintenance of said membrane.

Furthermore, it is envisaged that the nucleic acid molecule of the invention encodes a polypeptide, wherein said polypeptide contributing to membrane stability and/or function in organelles is a transporter molecule and/or a regulator of a transporter molecule. It is, e.g., envisaged that the polypeptide encoded by the nucleic acid molecule of the invention regulates, directly or indirectly, carrier and/or transport molecules capable of transporting molecules like ions, metabolites or vitamins across membranes and/or that said polypeptide is such a transporter/carrier molecule.

It is particularly preferred that the nucleic acid molecule the invention encodes a polypeptide as defined herein above, wherein said polypeptide is a modifying polypeptide. Particularly preferred modifying polypeptides comprise modifiers of mitochondrial proteins, for example the modification of a member of the UCP family.

Said member(s) of the UCP (uncoupling protein) family are known in the art and comprise, UCP1, UCP2, UCP3, UCP4, UCP5, StUCP or AtUCP, see, inter alia, Ricquier (2000), loc. cit. The above mentioned modification of mitochondrial proteins, and in particular of UCPs, may occur by direct interaction with said protein or, also, by supplying/importing/exporting ions, metabolites or vitamins and the like (or by blocking these processes) which are necessary for the function or activity of said mitochondrial protein or which are generated by the activity of said mitochondrial protein. Therefore, said “modification” also relates to transport- and supply-phenomena. Furthermore, said “modification” comprises the control of the function of one or more proteins/polypeptides, preferably of members of the UCP family. Most preferred are “modifications” comprising events which influence the metabolism of the cell, in particular the energy metabolism.

The present invention relates also, as pointed out herein above, to “variants” of the nucleic acid molecules described herein. The term “variant” means in this context that the nucleotide and their encoded amino acid sequence, respectively, of these polynucleotides differs from the sequences of the above-described nucleic acid molecules and (poly)peptides contributing to membrane stability and/or function of organelles in one or more nucleotide positions and are highly homologous to said nucleic acid molecules. Homology is understood as defined herein above. The deviations from the sequences of the nucleic acid molecules described above can, for example, be the result of nucleotide substitution(s), deletion(s), addition(s), insertion(s) and/or recombination(s). Homology can further imply that the respective nucleic acid molecules or encoded proteins are functionally and/or structurally equivalent. The nucleic acid molecules that are homologous to the nucleic acid molecules described above and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other mammals or mutations. The term “variants” in this context furthermore comprises, inter alia, allelic variations or splice variants as described herein above. Naturally occurring Unc-51, ROMA1, and/or 2TM protein variants or Unc-51, ROMA1, and/or 2TM gene variants are called “allelic variants”, and refer to one of several alternate forms of a gene occupying a given locus on a chromosome of an organism. (Genes II, Lewin, B., ed., John Wiley & Sons, New York (1985) and updated versions). These allelic variants can vary at either the polynucleotide and/or (poly)peptide level. Alternatively, non-naturally occurring variants may be produced by mutagenesis techniques or by direct synthesis. Using known methods of protein engineering and recombinant DNA technology, variants may be generated to improve or alter the characteristics of the herein described Unc-51, ROMA1, and/or 2TM proteins or Unc-51, ROMA1, and/or 2TM genes. Therefore, the term “allelic variant” also comprises synthetically produced or genetically engineered variants. The nucleic acid molecule of the invention may be of natural origin, synthetic or semisynthetic or it may be a derivative.

The nucleic acid molecules of the invention encoding the above described (poly)peptides, e.g. wildtype and mutated forms of Unc-51, ROMA1, and/or 2TM and/or fragments thereof find a wide variety of applications including use as translatable transcripts, hybridization probes, PCR primers or the use in expression profiling of nucleic acids, for example on appropriately coated chips or in diagnostic and/or pharmaceutical settings. Useful PCR primers can be deduced by the person skilled in the art from the nucleic acid molecules of the invention. Particularly useful primers are, inter alia, the employed in the appended examples.

In particular they may be used in detecting the presence of Unc-51, ROMA1, and/or 2TM genes and gene transcripts and in detecting and/or amplifying nucleic acids encoding further Unc-51, ROMA1, and/or 2TM homologues or structural analogues. Given the probes, materials and methods disclosed herein, inter alia, for probing cDNA and genomic libraries, the person skilled in the art is in a position to recover corresponding homologues. As described herein below, the nucleic acid molecules of the invention may be part of specific expression vectors and may be incorporated into recombinant cells for expression and screening and in transgenic animals for functional studies (e.g. the efficacy of candidate drugs for disease associated with expression of Unc-51, ROMA1, and/or 2TM) as described herein below.

Furthermore, in diagnosis, specific hybridization probes related to the Unc-51, ROMA1, and/or 2TM gene(s) as described herein and single nucleotide polymorphisms present in Unc-51, ROMA1, and/or 2TM alleles find use in identifying wild-type and mutant Unc-51, ROMA1, and/or 2TM alleles in clinical and laboratory samples. Mutant alleles are, inter alia, used to generate allele-specific oligonucleotide (ASO) probes for, e.g., high-throughput clinical diagnosis. For therapeutic approaches nucleic acid molecules of the invention as described herein above and herein below may be employed to modulate cellular expression or intracellular concentration or availability of active (poly)peptides of the invention. These nucleic acid molecules may comprise antisense molecules, i.e. single-stranded sequences comprising the complements of the disclosed nucleic acids of the invention.

The nucleic acid molecule(s) of the invention may be a recombinantly produced chimeric nucleic acid molecule comprising any of the aforementioned nucleic acid molecules either alone or in combination. Preferably, said nucleic acid molecule is part of a vector. The present invention therefore also relates to a vector comprising the nucleic acid molecule of the present invention.

The vector of the present invention may be, e.g., a plasmid, cosmid, virus, bacteriophage or another vector used e.g. conventionally in genetic engineering, and may comprise further genes such as marker genes which allow for the selection of said vector in a suitable host cell and under suitable conditions.

Furthermore, the vector of the present invention may, in addition to the nucleic acid sequences of the invention, comprise expression control elements, allowing proper expression of the coding regions in suitable hosts. Such control elements are known to the artisan and may include a promoter, a splice cassette, translation initiation codon, translation and insertion site for introducing an insert into the vector. Preferably, the nucleic acid molecule of the invention is operatively linked to said expression control sequences allowing expression in eukaryotic or prokaryotic cells.

Control elements ensuring expression in eukaryotic and prokaryotic cells are well known to those skilled in the art. As mentioned herein above, they usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Once the vector has been incorporated into the appropriate host, the host is maintained under conditions suitable for high level expression of the nucleotide sequences, and, as desired, the collection and purification of the (poly)peptide(s) or fragments thereof of the invention may follow.

Furthermore, the vector of the present invention may also be a gene transfer or gene targeting vector. Gene therapy, which is based on introducing therapeutic genes into cells by ex-vivo or in-vivo techniques is one of the most important applications of gene transfer. Suitable vectors, methods or gene-delivering systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813, Isner, Lancet 348 (1996), 370-374; Muhlhauser, Circ. Res. 77 (1995), 1077-1086; Onodua, Blood 91 (1998), 30-36; Verzeletti, Hum. Gene Ther. 9 (1998), 2243-2251; Verma, Nature 389 (1997), 239-242; Anderson, Nature 392 (Supp. 1998), 25-30; Wang, Gene Therapy 4 (1997), 393-400; Wang, Nature Medicine 2 (1996), 714-716; WO 94/29469; WO 97/00957; U.S. Pat. No. 5,580,859; U.S. Pat. No. 5,589,466; U.S. Pat. No. 4,394,448 or Schaper, Current Opinion in Biotechnology 7 (1996), 635-640, and references cited therein. In particular, said vectors and/or gene delivery systems are also described in gene therapy approaches in adipocyte (see, inter alia, U.S. Pat. No. 5,869,037 or Zhou, PNAS USA96 (1999), 2391-2395) or in the hypothalamus (see, inter alia, Geddes, Front Neuroendocrinol. 20 (1999), 296-316 or Geddes, Nat. Med. 3 (1997), 1402-1404). The nucleic acid molecules and vectors of the invention may be designed for direct introduction or for introduction via liposomes, viral vectors (e.g. adenoviral, retroviral), electroporation, ballistic (e.g. gene gun) or other delivery systems into the cell. Additionally, a baculoviral system can be used as eukaryotic expression system for the nucleic acid molecules of the invention.

As will be discussed herein below, the nucleic acid molecule of the present invention and/or the above described vectors/hosts of the present invention may be particularly useful as pharmaceutical compositions. Said pharmaceutical compositions may be employed in gene therapy approaches. In this context, it is envisaged that the nucleic acid molecules and/or vectors of the present invention may be employed to modulate, alter and/or modify the cellular expression and/or intracellular concentration of the (poly)peptide(s) of the invention or of (a) fragment thereof. Said modulation, alteration and/or modification may lead to up- or downregulation of the Unc-51, ROMA1, and/or 2TM (poly)peptide and/or the gene product of the herein described Unc-51, ROMA1, and/or 2TM gene. Furthermore, said therapeutic approache(s) may lead to an alteration and/or modulation of the availability of active Unc-51, ROMA1, and/or 2TM (poly) peptide/protein/gene product. In this context, the term “active” refers to the ability to perform its (normal) cellular function in an organism.

For gene therapy applications, nucleic acids encoding the (poly)peptide of the invention or fragments thereof may be cloned into a gene delivering system, such as a virus and the virus used for infection and conferring disease ameliorating or curing effects in the infected cells or organism.

As mentioned herein above, the nucleic acid molecule(s) and/or vector(s) may be employed in order to modulate/alter the gene expression or intracellular concentration of Unc-51, ROMA1, and/or 2TM protein/(poly)peptide. Said modulation/alteration may also be achieved by antisense-approaches.

Antisense modulation of Unc-51, ROMA1, and/or 2TM expression may employ antisense nucleic acids operably linked to gene regulatory sequences. For example, cells are transfected with a vector comprising an Unc-51, ROMA1, and/or 2TM sequence with a promoter sequence oriented such that transcription of the gene yields an antisense transcript capable of binding to endogenous Unc-51, ROMA1, and/or 2TM encoding mRNA. Transcription of the antisense nucleic acid may be constitutive or inducible and the vector may provide for stable extrachromosomal maintenance and integration. Alternatively, single-stranded antisense nucleic acids that bind to genomic DNA or mRNA encoding a (poly)peptide of the invention or a fragment thereof may be administered to the target cell, in or temporarily isolated from a host, at a concentration that results in a substantial reduction in expression of said (poly)peptide. Furthermore, it is envisaged that expression of the (poly)peptide of the invention may be influenced, e.g. suppressed by other means than antisense approaches. Therefore, reduced expression of the (poly)peptide of the invention may also be achieved by RNA-mediated gene interference, which applies double-stranded RNA instead of antisense nucleic acids (see, Sharp, Genes Dev. 13 (1999), 139-141). Gene suppression by double stranded RNA or RNAi-approach is also described in Hunter, Curr. Biol. 10 (2000), R137-R140.

The nucleic acid molecule of the invention may therefore be used for the construction of appropriate anti-sense oligonucleotides which are able to inhibit the function of the nucleic acid molecules which either encode wildtype or mutant versions of the Unc-51, ROMA1, and/or 2TM (poly)peptide of this invention. Said anti-sense nucleotide comprises preferably at least 15 nucleotides, more preferably at least 20 nucleotides, even more preferably 30 nucleotides and most preferably at least 40 nucleotides.

In addition, ribozyme approaches are also envisaged in this invention. Ribozymes may specifically cleave the nucleic acid molecule of the invention. In the context of the present invention ribozymes comprise, inter alia, hammerhead ribozymes, hammerhead ribozymes with altered core sequences or deoxyribozymes (see, e.g., Santoro, Proc. Natl. Acad. Sci. USA 94 (1997), 4262) and may comprise natural and in vitro selected and/or synthesized ribozymes.

Nucleic acid molecules according to the present invention which are complementary to nucleic acid molecules coding for proteins/(poly)peptides regulating, causing or contributing to obesity and/or encoding a mammalian (poly)peptide involved in the regulation of body weight (see herein below) may be used for the construction of appropriate ribozymes (see, e.g., EP-B1 0 291 533, EP-A1 0 321 201, EP-A2 0 360 257) which specifically cleave nucleic acid molecules of the invention. Selection of the appropriate target sites and corresponding ribozymes can be done as described for example in Steinecke, Ribozymes, Methods in Cell Biology 50, Galbraith, eds. Academic Press, Inc. (1995), 449-460.

The present invention also relates to a host cell transfected or transformed with the vector of the invention or a non-human host carrying the vector of the present invention, i.e. to a host cell or host which is genetically modified with a nucleic acid molecule according to the invention or with a vector comprising such a nucleic acid molecule. The term “genetically modified” means that the host cell or host comprises in addition to its natural genome a nucleic acid molecule or vector according to the invention which was introduced into the cell or host or into one of its predecessors/parents. The nucleic acid molecule or vector may be present in the genetically modified host cell or host either as an independent molecule outside the genome, preferably as a molecule which is capable of replication, or it may be stably integrated into the genome of the host cell or host.

The host cell of the present invention may be any prokaryotic or eukaryotic cell. Suitable prokaryotic cells are those generally used for cloning like E. coli or Bacillus subtilis. Furthermore, eukaryotic cells comprise, for example, fungal or animal cells. In a more preferred embodiment the host cell which is transformed with the vector of the invention is an adipose cell, a brain cell, a hepatic cell, an epithelial cell, a pancreatic cell, a blood cell or a cell (line) derived therefrom.

Hosts may be non-human mammals, most preferably mice, rats, sheep, calves, dogs, monkeys or apes and may comprise Psammomis obesus. Said mammals may be indispensable for developing a cure, preferably a cure for obesity, adipositas, eating disorders and/or disorders leading to a pathological body mass/body weight, pancreatic dysfunctions (for example diabetes), hypercholesterolemia, dyslipidemia, coronary heart disease, osteoarthritis, gallstones, cancers of the reproductive organs, sleep apnea, disorders related to ROS production, mitochondrial disorders, and others.

Furthermore, the hosts of the present invention may be partially useful in producing the (poly)peptides (or fragments thereof) of the invention. It is envisaged that said (poly)peptide (or fragments thereof) be isolated from said host.

The host of the present invention may also be a non-human transgenic animal as described herein below. The present invention also envisages non-human transgenic animals comprising a mutated form of the nucleic acid molecules of the invention or non-human transgenic animals wherein the nucleic acid molecule of the present invention has been deleted and/or inactivated. Said deletion may be a partial deletion. Particularly preferred non-human transgenic animals are Drosophila, Nematodes (like C. elegans), mice, rat, sheep and the like.

Furthermore, the present invention relates to a method of producing a (poly)peptide encoded by the nucleic acid molecule of the invention comprising culturing the host cell of the present invention under suitable conditions that allow the synthesis of said (poly)peptide and recovering and/or isolating the (poly)peptide produced from the culture.

Additionally, the present invention relates to a (poly)peptide encoded by the nucleic acid molecule of the invention or produced by or obtainable by the above-described method. The term “(poly)peptide” as employed herein denotes either a peptide, a full-length protein or (a) fragment(s) thereof. A peptide is preferably a fragment of the (poly)peptide of the invention. The term “(poly)peptide comprises (a) peptide(s) or (a) (poly)peptide(s) which encompass amino acid chains of any length, wherein the amino acid residues are linked by covalent peptide bonds. Preferably, said amino acid chains of a “peptide” comprise at least 10 amino acids, more preferably at least 20, more preferably at least 30, more preferably at least 40, even more preferably at least 50 and, most preferably at least 60 amino acids. It is even more preferred that the (poly)peptides of the invention comprise at least 100, more preferred at least 200, more preferred at least 300, more preferred at least 400, more preferred at least 500, even more preferred at least 600 amino acids.

The term “or (a) fragment(s) thereof” as employed in the present invention and in context with (poly)peptides of the invention, comprises specific peptides, amino acid stretches of the (poly)peptides as disclosed herein. It is preferred that said “fragment(s) thereof” is/are functional fragment(s). The term “functional fragment” denotes a part of the above identified (poly)peptide of the invention which fulfils, at least in part, physiological and/or structural activities of the (poly)peptide of the invention. It is, however, also envisaged that said fragment functions as intervening and/or inhibiting molecule for the (poly)peptide of the invention. For example, it is envisaged that fragments of the (poly)peptide of the invention may structurally and/or physiologically interact with the (poly)peptide of the invention and thereby inhibit the function of said (poly)peptide.

The (poly)peptides of the present invention may be recombinant (poly)peptides expressed in host cells like bacteria, yeasts, or other eukaryotic cells, like mammalian or insect cells. Alternatively, they may be isolated from viral preparations. In another embodiment of the present invention, synthetic (poly)peptides may be used. Therefore, such a (poly)peptide may be a (poly)peptide as encoded by the nucleic acid molecule of the invention which only comprises naturally occurring amino acid residues, but it may also be a (poly)peptide containing modifications. The (poly)peptide of the present invention can be, for example, the product of expression of a nucleotide sequence encoding such a (poly)peptide, a product of chemical modification or can be purified from natural sources, for example, viral preparations. Furthermore, it can be the product of covalent linkage of (poly)peptide domains.

The peptides/(poly)peptides may also be produced by biochemical or synthetic techniques. Those methods are known to those of ordinary skill in the art (see, e.g. Merrifield, J. Am. Chem. Soc. 85 (1963), 2149-2146; Stewart, “Solid Phase Peptide Synthesis”, WH Freeman Co, San Francisco (1969); Scopes, “Protein Purification”, Springer Verlag, New York, Heidelberg, Berlin (1987); Janson, “Protein Purification, Principles, High Resolution Methods and Applications”, VCH Publishers, New York, Weinheim, Cambridge (1989); Wrede, “Concepts in Protein Engineering and Design”, Walter de Gruyter, Berlin, New York (1994)).

The present invention also relates to a fusion protein comprising the (poly)peptide of the invention or (a) fragment thereof. Therefore, in addition to the (poly)peptides of the present invention, said fusion protein can comprise at least one further domain, said domain being linked by covalent or non-covalent bonds. The linkage can be based on genetic fusion according to the methods known in the art (Sambrook et al., loc. cit., Ausubel, “Current Protocols in Molecular Biology”, Green Publishing Associates and Wiley Interscience, N.Y. (1989)) or can be performed by, e.g., chemical cross-linking as described in, e.g., WO 94/04686. The additional domain present in the fusion protein comprising the (poly)peptide of the invention may preferably be linked by a flexible linker, advantageously a (poly)peptide linker, wherein said (poly)peptide linker preferably comprises plural, hydrophilic, peptide-bonded amino acids of a length sufficient to span the distance between the C-terminal end of said further domain and the N-terminal end of the peptide, (poly)peptide or antibody or vice versa. The above described fusion protein may further comprise a cleavable linker or cleavage site, which, for example, is specifically recognized and cleaved by proteinases or chemical agents.

Additionally, said at least one further domain may be of a predefined specificity or function. In this context, it is understood that the (poly)peptides of the invention may be further modified by conventional methods known in the art. This allows for the construction of fusion proteins comprising the (poly)peptide of the invention and other functional amino acid sequences, e.g., organelle localization signals, transactivating domains, DNA-binding domains, hormone-binding domains, protein tags (e.g. GST, GFP, h-myc peptide, FLAG, HA peptide, Strep), transmembrane domains or fatty acid attachment motifs which may be derived from heterologous proteins.

The fusion protein of the invention may also be a mosaic (poly)peptide comprising at least two epitopes of the (poly)peptide of the invention wherein said mosaic (poly)peptide lacks amino acids normally intervening between the epitopes in the native Unc-51, ROMA1, and/or 2TM protein. Inter alia, such mosaic (poly)peptides are useful in the applications and methods described herein, since they may comprise within a single peptide or (poly)peptide a number of relevant epitopes possibly presented linearly or as multi-antigen peptide system in a case of lysines. Relevant epitopes can be separated by spacer regions.

The nucleic acid molecule, the (poly)peptide (as well as the antibody or fragment or derivative thereof, the aptamer or other receptor described herein), the fusion protein, the mosaic (poly)peptide or the anti-sense oligonucleotide of the invention may be detectably labeled. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. Detection methods comprise, but are not limited to, autoradiography, fluorescence microscopy, direct and indirect enzymatic reactions, etc.

The present invention furthermore additionally relates to an antibody or a fragment or derivative thereof or an antiserum or an aptamer or another receptor specifically recognizing an epitope on the nucleic acid, or the (poly)peptide of the invention. The general methodology for producing antibodies is well-known and has, for monoclonal antibodies, been described in, for example, Köhler and Milstein, Nature 256 (1975), 494 and reviewed in J. G. R. Hurrel, ed., “Monoclonal Hybridoma Antibodies: Techniques and Applications”, CRC Press Inc., Boco Raron, Fla. (1982). In accordance with the present invention the term “antibody” relates to monoclonal or polyclonal antibodies. Polyclonal antibodies (antiserum) can be obtained according to conventional protocols. Antibody fragments or derivatives comprise F(ab′)₂, Fab, Fv or scFv fragments; see, for example, Harlow and Lane, “Antibodies, A Laboratory Manual”, CSH Press 1988, Cold Spring Harbor, N.Y. Preferably the antibody of the invention is a monoclonal antibody. Furthermore, in accordance with the present invention, the derivatives of the invention can be produced by peptidomimetics. In the context of the present invention, the term “aptamer” comprises nucleic acids such as RNA, ssDNA (ss=single stranded), modified RNA, modified ssDNA or PNAs which bind a plurality of target sequences having a high specificity and affinity. Aptamers are well known in the art and, inter alia, described in Famulok, Curr. Op. Chem. Biol. 2 (1998), 320-327. The preparation of aptamers is well known in the art and may involve, inter alia, the use of combinatorial RNA libraries to identify binding sites (Gold, Ann. Rev. Biochem. 64 (1995), 763-797). Said other receptors may, for example, be derived from said antibody etc. by peptidomimetics. The specificity of the recognition implies that other known proteins, molecules are not bound. A suitable host for assessing the specificity would imply contacting the above recited compound comprising an epitope of the nucleic acid molecule or the (poly)peptide of the invention as well as corresponding compounds e.g. from protein or nucleic acid molecules known in the art, for example in an ELISA format and identifying those antibodies etc. that only bind to the compound of the invention but do not or to no significant extent cross-react with said corresponding compounds.

The invention also relates to an anti-sense oligonucleotide of a nucleic acid molecule of the invention. As said anti-sense oligonucleotide may be employed in scientific as well as in diagnostic or in therapeutic purposes.

The invention furthermore provides for a non-human animal expressing the polypeptide of the invention or the fusion protein of the invention or which is transfected with the vector of the invention which comprises the nucleic acid molecule of the invention. It is envisaged, for example, that the non-human animal over- or under-expresses the polypeptide of the invention. Furthermore, the invention relates to a non-human animal, wherein the nucleic acid molecule of the invention or a homolog, paralog or ortholog thereof is silenced and/or mutated.

The above mentioned non-human animal is preferably selected from the group consisting of mouse, rat, sheep, hamster, pig, dog, monkey, rabbit, calf, horse, nematodes, fly and fish. The invention also relates to transgenic non-human animals such as transgenic mice, rats, hamsters, dogs, monkeys, rabbits, pigs, C. elegans, Drosophila, fish (like zebrafish or torpedofish) comprising a nucleic acid molecule or vector of the invention. Said animal may have one or several copies of the same or different nucleic acid molecules encoding one or several forms of the (poly)peptide of the invention, regulating, causing or contributing to obesity or involved in the regulation of body weight. These animals are partially useful as research models for obesity, adipositas, eating disorders, wasting and/or other disorders of body weight/body mass, pancreatic dysfunctions (for example diabetes), hypercholesterolemia, dyslipidemia, coronary heart disease, osteoarthritis, gallstones, cancers of the reproductive organs, sleep apnea, disorders related to ROS production, and others, as described herein. Furthermore, said transgenic non-human animals are well suited for, e.g., pharmacological studies of drugs in connection with mutant forms of the above described Unc-51, ROMA1, and/or 2TM protein.

In another embodiment, the present invention relates to the use of the nucleic acid molecule, the vector, the host, the polypeptide, the fusion protein, the antibody, fragment or derivative thereof or an aptamer or another receptor or the anti-sense oligonucleotide of the invention for controlling the function of a gene and/or a gene product which is influenced and/or modified by a polypeptide as defined herein, e.g. Unc-51, ROMA1, and/or 2TM gene or protein.

Said influence/modification may occur by direct interaction between proteins/protein fragments and/or by providing metabolic compounds, or ions that are necessary for the function, activity and/or expression of said gene and/or gene product. It is particularly preferred that said gene and/or gene product is a gene and/or gene product expressed in organelles. Said organelle may be, inter alia, a mitochondrium or a peroxisome.

It is particularly preferred that said gene and/or gene product is a member of the UCP family. Members of the UCP family are well known and described herein above.

The present invention furthermore provides for a composition comprising the nucleic acid molecule, the vector, the host, the polypeptide, the fusion protein, the antibody, fragment or derivative thereof or an aptamer or another receptor or the anti-sense oligonucleotide of the invention. Said composition may be, inter alia, a diagnostic composition or a pharmaceutical composition.

In addition, the present invention provides for the use of the composition as defined herein for detecting and/or verifying an disorder in cells, cell masses, organs and/or subjects and/or for the treatment, alleviation and/or prevention of an disorder in cells, cell masses, organs and/or subjects. Said disorder may be a metabolic disorder or a mitochondrial disorder, whereby mitochondrial disorders comprise disorders like deafness, retinopathies, progressive encelopathies, ataxias, spastic paraplegia, metabolic acidosis and others. Said metabolic disorder may comprise obesity, adipositas, eating disorders (bulimia nervosa, anorexia nervosa), cachexia (wasting), pancreatic dysfunction (like diabetes, in particular type 2 diabetes) and/or a disorder related to ROS (reactive oxygen species) production (in particular responses to infections, in aging and cancerogenesis). For example, it has been shown that UCPs are involved in pancreatic disorders, e.g. diabetes. A role for uncoupling proteins in diabetes was demonstrated by induction of UCP3 in Streptozotocin-induced diabetes in rodents (see, inter alia, Hidaka, Proc Soc Exp Biol Med 224: 172-177 (2000), Hidaka, Diabetes 48: 430-435 (1999)). Furthermore it was shown that UCP2 expression in pancreatic beta-cells influences beta-cell function and insulin secretion (Wang, Diabetes 48: 1020-1025 (1999); Chan, Diabetes 48: 1482-1486 (1999)).

Reactive oxygen species (ROS) can lead to membrane dysfunction, DNA damage and inactivation of proteins. Pathological consequences include cancer, arthritis and neurodegenarative disease. ROS limiting metabolism is a major mechanism to protection from cellular damage. In particular obesity can cause increased oxidative stress (Hayes, Free Radic Res 31: 273-300 (1999); Yang, Arch Biochem Biophys 378: 259-268 (2000)). In contrast, increased ROS production in macrophages can improve immune response. So are UCP2 knockout mice more resistant against infection with certain pathogens. Therefore, the compounds of the present invention, being capable of modifying, inter alia, UCPs may be well suited for the above identified purposes.

In a further embodiment, the present invention relates to the use of the nucleic acid molecule, the vector, the host, the polypeptide, the fusion protein, the antibody, fragment or derivative thereof or an aptamer or another receptor or the anti-sense oligonucleotide for identifying substances capable of interacting with the polypeptide as defined in herein. Said substance is capable of interacting with said polypeptide may be (an) antagonist(s) or (an) agonist(s).

In yet a further embodiment, the present invention provides for a method of identifying a polypeptide or (a) substance(s) involved in cellular metabolism in an animal or capable of modifying homeostasis comprising the steps of:
(a) testing a collection of polypeptides or substances for interaction with the polypeptide of the invention or (a) fragment(s) thereof or the fusion protein of the invention or (a) fragment(s) thereof using a readout system; and
(b) identifying polypeptides or substances which test positive for interaction in step (a).

The term “cellular metabolism” as used herein above may comprise an metabolic event involved in the regulation of ion-, vitamin- or metabolite-transport across organelle membranes. These transport events or the regulation thereof may influence energy homeostasis, accumulation of storage compounds and/or radical production/elimination.

The polypeptide or substance identified by the method disclosed herein above may be, inter alia, a polypeptide or a substance interacting directly or indirectly (e.g. via linker proteins or via physiological parameters) with the polypeptide of the invention, i.e. with Unc-51, ROMA1, and/or 2TM proteins and/or a fragment thereof. Said testing for interaction of step (a) as described herein above may be carried out by methods known to the skilled artisan and were described herein. In particular these assays comprise biochemical, immunological and/or molecular biological assays.

Said interaction assays employing read-out systems are well known in the art and comprise, inter alia, two hybrid screenings (as, described, inter alia, in EP-0 963 376, WO 98/25947, WO 00/02911) GST-pull-down columns, co-precipitation assays from cell extracts as described, inter alia, in Kasus-Jacobi, Oncogene 19 (2000), 2052-2059, “interaction-trap” systems (as described, inter alia, in U.S. Pat. No. 6,004,746) expression cloning (e.g. lamda gtII), phage display (as described, inter alia, in U.S. Pat. No. 5,541,109), in vitro binding assays and the like. Further interaction assay methods and corresponding read out systems are, inter alia, described in U.S. Pat. No. 5,525,490, WO 99/51741, WO 00/17221, WO 00/14271 or WO 00/05410.

Similarly, interacting molecules/(poly)peptides may be deduced by cell-based techniques well known in the art. These assays comprise, inter alia, the expression of reporter gene constructs or “knock-in” assays, as described, for, e.g., the identification of drugs/small compounds influencing the gene expression. Said “knock-in” assays may comprise “knock-in” in tissue culture cells, as well as in (transgenic) animals. Examples for successful “knock-ins” are known in the art (see, inter alia, Tanaka, J. Neurobiol. 41 (1999), 524-539 or Monroe, Immunity 11 (1999), 201-212). Furthermore, biochemical assays may be employed which comprise, but are not limited to, binding of the (poly)peptides of the invention (or (a) fragment(s) thereof) to other molecules/(poly)peptides, peptides or binding of the (poly)peptides of the invention (or (a) fragment(s) thereof) to itself (themselves) (dimerizations, oligomerizations, multimerizations) and assaying said interactions by, inter alia, scintillation proximity assay (SPA) or homogenous time-resolved fluorescence assay (HTRFA).

Said “testing of interaction” may also comprise the measurement of a complex formation. The measurement of a complex formation is well known in the art and comprises, inter alia, heterogeneous and homogeneous assays. Homogeneous assays comprise assays wherein the binding partners remain in solution and comprise assays, like agglutination assays. Heterogeneous assays comprise assays like, inter alia, immuno assays, for example, ELISAs, RIAs, IRMAs, FIAs, CLIAs or ECLs.

Further methods and assays for identifying interaction and/or binding partners of the (poly)peptides of the invention or for the identification of agents/compounds which are capable of interfering with the binding of the (poly)peptides of the invention with this (specific) intracellular binding partners/targets are disclosed herein below. Said additional and/or further method(s) and assay(s) may also be employed in the above described method for identifying a (poly)peptide involved in the regulation of body weight and/or capable of interacting with the Unc-51, ROMA1, and/or 2TM (poly)peptide of the invention.

Any measuring or detection step of the method(s) of the present invention may be assisted by computer technology. For example, in accordance with the present invention, said detection and/or measuring step can be automated by various means, including image analysis, spectroscopy or flow cytometry.

In yet another embodiment, the present invention relates to the method(s) described herein above, which further comprises the step of identifying the nucleic acid molecule(s) encoding the one or more interacting (poly)peptides.

The identification of such nucleic acid molecule(s) is well known in the art and comprises, inter alia, the use of specific and/or degenerate primers. Furthermore, recombinant technologies as described in Sambrook, loc. cit. or in Glick (1994), “Molecular Biotechnology”, ASM Press, Washington may be employed.

In yet a further embodiment, the present invention relates to a method of identifying a polypeptide or (a) substance(s) involved in cellular metabolism in an animal or capable of modifying homeostasis comprising the steps of
(a) testing a collection of polypeptides or substances for interaction with the polypeptide of the invention or identified by the method described herein above; and
(b) identifying polypeptides that test positive for interaction in step (a); and optionally
(c) repeating steps (a) and (b) with the polypeptides identified one or more times wherein the newly identified polypeptide replaces the previously identified polypeptide as a bait for the identification of a further interacting polypeptide.

The methods described herein above may further comprise the step of identifying the nucleic acid molecule(s) encoding the one or more interacting (poly)peptides.

The present invention also provides for the use of nucleic acid molecules as described herein or of polypeptides as described herein for the detection and/or isolation of genes and/or gene products involved in functional cascades of cell metabolism, in particular of energy metabolism.

Additionally, the present invention relates to a method of identifying a polypeptide involved in the regulation of body weight in a mammal comprising the steps of
(a) contacting a collection of (poly)peptides with the polypeptide of the invention or (a) fragment(s) thereof or the fusion protein of the invention or (a) fragment(s) thereof under conditions that allow binding of said (poly)peptides;
(b) removing (poly)peptides from said collection of (poly)peptides that did not bind to said polypeptide of the invention or the fusion protein of the invention in step (a); and
(c) identifying (poly)peptides that bind to said polypeptide or the fusion protein of the invention.

The method as described herein above may be carried out by the person skilled in the art without further ado. Said “contacting” of step (a) may, inter alia, be carried out in solution employing (magnetic) beads coupled with the (poly)peptide of the invention and/or fragments thereof. Non-bound (poly)peptides may be easily removed by methods known in the art, comprising, for example, magnetic separation, gravity, affinity column systems and corresponding washes and the like.

Methods for identifying bound (poly)peptides are well known in the art and comprise, inter alia, SDS PAGE analysis and Western blotting. Furthermore, techniques like 2D-gel electrophoresis, in-gel digests, microsequencing, N-terminal sequencing, MALDI-MS, analysis of peptides in mass spectroscopy, peptide mass fingerprinting, PSD-MALDI-MS and/or (micro-) HPLC. Separated polypeptdies to be identified may be further analyzed by, inter alia, Edman-degradation, MALDI-MS methods, ladder sequencing (Thiede, FEBS 357 (1995), 65).

By use of the above described and mentioned methods (and others known in the art) amino acid sequences of the (poly)peptides to be identified can be deduced and sequenced. From these sequenced amino acid fragments, degenerative oligonucleotides may be deduced and synthesized that may be used to screen, for example, genomic or cDNA libraries to identify and clone the corresponding gene/cDNA.

Furthermore, phage display approaches may be employed in the method(s) of this invention. Phage display allows the identification of proteins that interact with a molecule of interest. Libraries of phage, each displaying a different peptide epitope are tested for binding to the molecule of interest. Bound phages can be purified and the insert encoding the peptide epitope may be sequenced. Phage display kit(s) are known in the art and commercially available, e.g., Display Systems Biotech Cat. No. 300-110.

The present invention relates, in yet another embodiment to the method(s) described herein, wherein said (poly)peptide of the invention is fixed to a solid support. Solid supports are well known in the art and comprise, inter alia, commercially available column materials, polystyrene beads, latex beads, magnetic beads, colloid metal particles, glass and/or silicon chips and surfaces, nitrocellulase strips, membranes, sheets, duracytes, wells and walls of reaction trays, plastic tubes etc. Suitable methods for fixing/immobilizing said (poly)peptide(s) of the invention are well known and include, but are not limited to ionic, hydrophobic, covalent interactions and the like. In a particular preferred embodiment, said solid support is a gel filtration or an affinity chromatography material.

In a yet more preferred embodiment of the method of the invention as described herein above, said binding (poly)peptides are released prior to said identification in step (c). Said release may be effected by elution. Such elution methods are well known in the art and comprise, inter alia, elution with solutions of different ionic strength or different pH, or with intercalating or competing agents/molecules/peptides.

Furthermore, in a yet more preferred embodiment, the present invention relates to the above described method of the invention, wherein said method further comprises the step of identifying the nucleic acid molecule(s) encoding the one or more binding (poly)peptides.

As pointed out herein above, said nucleic acid molecule(s) may be deduced, inter alia, by employing degenerate primers/oligonucleotides in order to detect the corresponding gene(s) and/or cDNA(s) or by expression cloning.

A method of identifying a compound influencing the expression of the nucleic acid molecule of the invention comprising the steps of
(a) contacting a host carrying an expression vector comprising the nucleic acid molecule of the invention or the nucleic acid molecule identified by the method of the invention operatively linked to a readout system with a compound or a collection of compounds;
(b) assaying whether said contacting results in a change of signal intensity provided by said readout system; and, optionally,
(c) identifying a compound within said collection of compounds that induces a change of signal in step (b);
   wherein said change in signal intensity correlates with a change of expression of said nucleic acid molecule.

Furthermore, the present invention provides for a method of identifying a compound influencing the activity of a polypeptide as defined herein above comprising the steps of
(a) contacting a host carrying an expression vector comprising the nucleic acid molecule of the invention operatively linked to a readout system and/or carrying a (poly)peptide of the invention linked to a readout system with a compound or a collection of compounds;
(b) assaying whether said contacting results in a change of signal intensity provided by said readout system; and, optionally
(c) identifying a compound within said collection of compounds that induces a change of signal in step (b);
   wherein said change in signal correlates with a change in activity of said (poly)peptide.

The term “activity” as used herein above in context of the method of the invention also comprises the “function” of (a) (poly)peptide(s) of the invention. Said function may comprise, as mentioned herein above, enzymatic activities or other functions, like, inter alia, involvement in signalling pathways. Such activities and modulators of such activities may be determined and/or identified by convenient in vitro or in vivo assays as described herein or by variations thereof. The underlying technology is widely and commonly known to the person skilled in the art.

Readout systems operatively linked to the nucleic acid molecules of the invention or linked to the (poly)peptides of the invention are disclosed herein and comprise, but are not limited to, assays based on radioactive labels, luminescence, fluorescence, etc. Inter alia, said readout system may comprise fluorescence resonance energy transfer (FRET). The above described methods are particularly useful in (automated) high-throughput screenings. In context of this invention, the above mentioned “readout system opertatively linked to the nucleic acid molecules of the invention” also comprises readout systems which are located on different molecules, e.g. nucleic acid molecules, like, inter alia, other plasmids, vectors etc. Said host of step (a) of the methods described herein above may be a eukaryotic host cell. Said host cell may be a yeast cell or a plant cell. It is particularly preferred that said eukaroytic host cell is a mammalian host cell. However, said host cell may also be a prokaryotic cell, e.g. a bacterium. Particularly preferred are prokaryotic (host) cells as described herein above.

The term “compound” in the method(s) of the invention includes a single substance or a plurality of substances which may or may not be identical. Said compound(s) may be comprised in, for example, samples, e.g., cell extracts from, e.g., plants, animals or microorganisms. The compound (substance) may be a peptide or a low-molecular weight organic molecule which may be derived from a compound library which is screened, e.g. by a method as described herein. Furthermore, said compound(s) may be known in the art but hitherto not known to be capable of influencing the activity of (a) (poly)peptide(s) of the invention or not known to be capable of influencing the expression of the nucleic acid molecule of the invention, respectively. The plurality of compounds may be, e.g., added to a sample in vitro, to the culture medium or injected into the cell.

If a sample (collection of compounds) containing (a) compound(s) is identified in the method(s) of the invention, then it is either possible to isolate the compound from the original sample identified as containing the compound in question or one can further subdivide the original sample, for example, if it consists of a plurality of different compounds, so as to reduce the number of different substances per sample and repeat the method with the subdivisions of the original sample. It can then be determined whether said sample or compound displays the desired properties by methods known in the art such as described herein. Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art (see, e.g., EP-A-0 403 506). Furthermore, the person skilled in the art will readily recognize which further compounds and/or cells may be used in order to perform the methods of the invention, for example, host cells as described herein above or enzymes, if necessary, that, e.g., convert a precursor compound into the active compound which in turn influences the expression of the nucleic acid molecule of the invention and/or influences the activity of (a) (poly)peptide of the invention. Such adaptation of the method of the invention is well within the skill of the person skilled in the art and can be performed without undue experimentation.

Compounds which can be used in accordance with the method of the present invention include, inter alia, peptides, proteins, nucleic acids including cDNA expression libraries, antibodies, small organic compounds, ligands, PNAs and the like. Said compounds can also be functional derivatives or analogues of known activators or inhibitors. Methods for the preparation of chemical derivatives and analogues are well known to those skilled in the art and are described in, for example, Beilstein, loc. cit. Furthermore, said derivatives and analogues can be tested for their effects according to methods known in the art and/or as described herein. Furthermore, peptidomimetics and/or computer aided design of appropriate activators or inhibitors of the expression of the nucleic acid molecules of the invention or of the activity of (a) (poly)peptide of the invention can be used, for example, according to the methods described herein. Appropriate computer systems for the computer aided design of, e.g., proteins and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. The results obtained from the above-described computer analysis can be used in combination with the method of the invention for, e.g., optimizing known compounds, substances or molecules. Appropriate compounds can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of inhibitors or activators of Unc-51, ROMA1, and/or 2TM protein or the Unc-51, ROMA1, and/or 2TM nucleic acid molecule can be used for the design of peptidomimetic inhibitors or activators of the (poly)peptide of the invention to be tested in the method of the invention (Rose, Biochemistry 35 (1996), 12933-12944; Rutenber, Bioorg. Med. Chem. 4 (1996), 1545-1558).

In yet a further embodiment, the invention provides for a method of assessing the impact of the expression of one or more polypeptides or of one or more fusion proteins of the invention in a non-human animal comprising the steps of
(a) overexpressing the nucleic acid molecule of the invention or the nucleic acid molecule of the invention in said animal; and
(b) determining whether the weight of said animal has increased, decreased, whether metabolic changes are induced and/or whether the eating behaviour is modified.

Similarly, the present invention also relates to a method of assessing the impact of the expression of one or more (poly)peptides or of one or more fusion proteins of the invention in a non-human animal comprising the steps of
(a) underexpressing the nucleic acid molecule of the invention in said animal; and
(b) determining whether the weight of said animal has increased or decreased, whether metabolic changes are induced and/or whether the eating behaviour is modified.

Transgenic non-human animals as described herein above may be particularly useful for the above described methods of assessing the impact of the expression of one or more (poly)peptide of the invention. The above mentioned “underexpression” of the nucleic acid molecule of the invention comprises, inter alia, full deletions of both alleles, or the deletion of any one allele. Furthermore, said term comprises the generation of a mutation which leads to the expression of a less functional protein/(poly)peptide in the test animal.

A method of screening for an agent which modulates the interaction of a polypeptide as defined herein above with a binding target/agent, comprising the steps of
(a) incubating a mixture comprising
   (aa) a polypeptide of the invention, or a fragment thereof or a fusion protein of the invention or a fragment thereof;
   (ab) a binding target/agent of said (poly)peptide or fusion protein or fragment thereof; and
   (ac) a candidate agent
   under conditions whereby said (poly)peptide, fusion protein or fragment thereof specifically binds to said binding target/agent at a reference affinity;
(b) detecting the binding affinity of said (poly)peptide, fusion protein or fragment thereof to said binding target to determine an (candidate) agent-biased affinity; and
(c) determining a difference between (candidate) agent-biased affinity and the reference affinity.

As pointed out herein above, a specific binding target/agent of the (poly)peptide(s) of the present invention may comprise molecules involved in signalling pathways and/or specific receptors contacting of the (poly)peptide of the invention. However, it is also envisaged that said binding target/agent of the (poly)peptide of the invention is said (poly)peptide itself, leading, inter alia, to dimerizations or multimerizations. Further (natural and artificial) binding targets/agents may be identified by methods known in the art and disclosed herein.

The “reference affinity” of the interaction of the (poly)peptides of the invention and its binding targets/agents may be established and/or deduced by methods known in the art. Said methods comprise, but are not limited to, in vitro and in vivo methods and may involve binding assays as described herein. In particular, said binding assays encompass any assay where the molecular interaction of the (poly)peptides of the invention with binding targets/agents be evaluated. Said binding target/agent may comprise natural (e.g. intracellular) binding targets/agents, such as, e.g., Unc-51-, ROMA1- and/or 2TM-substrate, Unc-51, ROMA1, and/or 2TM (poly)peptide itself, Unc-51, ROMA1, and/or 2TM (poly)peptide regulators and/or molecules of signalling cascades. Within the scope of this invention are, however also non-natural binding partners of the (poly)peptide of the invention, which may comprise, e.g., antibodies or derivatives and/or fragments thereof, aptamers, as well as non-natural receptor molecules. Said binding targets/agents also comprise antagonists as well as agonists of the (poly)peptides of the present invention.

Specific affinities, activities and/or function of the (poly)peptide(s) of the invention may be determined by convenient in vitro, cell-based or in vivo assays, e.g. in vitro binding assays, cell culture assays, in animals (e.g. gene therapy, transgenics), etc. Binding assays encompass any assay where the molecular interaction of a (poly)peptide of the invention with a binding target is evaluated. The binding target may be a natural intracellular binding target such as oligomerization (dimerization, multimerization) of said (poly)peptide of the invention itself, a substrate or a regulating protein of said (poly)peptide of the invention or another regulator that directly modulates the activity or the (cellular) localization of the (poly)peptides of the invention. Further binding targets/agents comprise non-natural binding targets like (a) specific immune protein(s) such as an antibody, or an Unc-51, ROMA1, and/or 2TM (poly)peptide specific agent such as those identified in screening assays as described below.

Specific screening assays are, inter alia, disclosed in U.S. Pat. No. 5,854,003 or in U.S. Pat. No. 5,639,858. Specific binding agents of the (poly)peptides of the invention may include Unc-51-, ROMA1- and/or 2TM-specific receptors, such as those of the family of heptahelical receptors. Other natural Unc-51, ROMA1, and/or 2TM binding targets are readily identified by screening cells, membranes and cellular extracts and fractions with the disclosed materials and methods and by other methods known in the art. For example, natural intracellular binding targets of the (poly)peptide of the invention may be identified with assays such as one-, two-, and three-hybrid screens. In addition, biochemical purification procedures, co-precipitation assays from cell extracts, interaction-trap” systems, expression cloning (e.g. in bacteria using lambda gt11 or in eukaryotic cell systems using plasmid expression vectors), phage display, and the like, may be utilised for identification of natural Unc-51, ROMA1, and/or 2TM binding agents. Non-natural intracellular binding agents may be obtained in screens of chemical libraries such as described below, etc.

The invention provides efficient methods of identifying pharmacological agents, compounds or lead compounds for agents active at the level of Unc-51, ROMA1, and/or 2TM modulatable cellular function. Generally, these screening methods involve assaying for compounds, which modulate interaction of the (poly)peptides of the invention with a natural Unc-51, ROMA1, and/or 2TM binding target. A wide variety of assays for binding agents are provided including labeled in vitro protein-protein binding assays, immunoassays, cell based assays, etc. The methods are amenable to automated, cost-effective high-throughput screening of chemical libraries for lead compounds and have immediate application in a broad range of domestic and international pharmaceutical and biotechnology drug development programs. Identified reagents find use in the pharmaceutical industries for animal and human trials; for example, the reagents may be derivatised and rescreened in vitro and in vivo assays to optimise activity and minimise toxicity for pharmaceutical development.

In vitro binding assays employ a mixture of components including a (poly)peptide of the invention, which may be part of a fusion product with another peptide or (poly)peptide(s), e.g. a tag for detection or anchoring, etc. The (poly)peptides of the invention or fragment(s) thereof used in the methods are usually added in an isolated, partially pure or pure form and are typically recombinantly produced. The assay mixture also comprises a candidate pharmacological agent at different concentrations. Candidate agents encompass numerous chemical classes, though typically they are organic compounds; preferably small organic compounds. Small organic compounds have a molecular weight of more than 50 Da yet less than about 2,500 Da, preferably less than about 1,000 Da, more preferably, less than about 500 Da. Candidate agents comprise functional chemical groups necessary for structural interactions with proteins and/or DNA, and typically include at least an amine, carbonyl, hydroxyl or carboxyl group, preferably at least two of the functional chemical groups, more preferably at least three. The candidate agents often comprise cyclical carbon or heterocyclic structures and/or aromatic or polyaromatic structures substituted with one ore more of the aforementioned functional groups. Candidate agents are also found among biomolecules including peptides, saccharides, fatty acids, steroids, purine, pyrimidies, derivatives, structural analogues or combinations thereof, and the like. Where the agent is or is encoded by a transfected nucleic acid, said nucleic acid is typically DNA or RNA.

Candidate agents are obtained from a wide variety of sources including libraries of synthetic or natural compounds. For example, numerous means are available for random and directed synthesis of a wide variety of organic compounds and biomolecules. Alternatively, libraries of natural compounds in the form of bacterial, fungal, plant, and animal extracts are available or readily produced. Additionally, natural and synthetically produced libraries and compounds are readily modified through conventional chemical, physical, and biochemical means. In addition, known pharmacological agents may be subject to directed or random chemical modifications to produce structural analogues.

A variety of other reagents may also be included in the mixture. These include reagents required as biochemical energy sources, e.g. ATP or ATP analogues, nucleic acids, e.g. in nucleic acids binding assays, salts, buffers, neutral proteins, e.g. albumin, detergents, etc., which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

The resultant mixture is incubated under conditions whereby, but for the presence of the candidate pharmacological agent, the Unc-51, ROMA1, and/or 2TM polypeptide specifically binds the cellular binding target, portion or analogue with a reference binding affinity. The mixture components can be added in any order that provides for the requisite binding and incubations may be performed at any temperature, which facilitates optimal binding. Incubation periods are likewise selected for optimal binding but also minimised to facilitate rapid, high-throughput screening. Generally a plurality of assay mixtures are run in parallel with different agent concentrations to obtain a differential response to the various concentrations. Typically, one of these concentrations serves as a negative control, i.e. at zero concentration or below the limits of assay detection.

After incubation, the agent-biased binding and/or affinity between the (poly)peptide of the invention and one or more binding targets is detected by any convenient way. For cell-free binding type assays, a separation step is often used to separate bound from unbound components. The separation step may be accomplished in a variety of ways. Conveniently, at least one of the components is immobilised on a solid substrate, which may be any solid from which the unbound components may be conveniently separated. The solid substrate may be made of a wide variety of materials and in a wide variety of shapes, e.g. microtiter plate, microbead, dipstick, resin particle, etc. The substrate is chosen to maximise the signal to noise ratios, primarily to minimise background binding, for ease of washing and cost.

Separation may be effected for example, by removing a bead or a dipstick from a reservoir, emptying or diluting a reservoir such as a microtiter plate well, rinsing a bead (e.g. beads with iron cores may be readily isolated and washed using magnets), particle, chromatographic column or filter with a wash solution or solvent. Typically, the separation step will include an extended rinse or wash or a plurality of rinses and washes. For example, where the solid substrate is a microtiter plate, the wells may be washed several times with a washing solution, which typically includes those components of the incubation mixture that do not participate in specific binding such as salts, buffer, detergent, non-specific protein, etc.

Alternatively, cell-free binding type assays may be performed in homogeneous formats that do not require a separation step, e.g. scintillation proximity assay (SPA), homogenous time-resolved fluorescence assay (HTRFA). Further methods which may be employed comprise fluorescence polarisation (FP) and fluorescence resonance energy transfer (FRET).

Detection may be effected in any convenient way. For cell based assays such as one, two, and three hybrid screens, the transcript resulting from Unc-51-target binding usually encodes a directly or indirectly detectable product (e.g. galactosidase activity, luciferase activity, etc.). For cell-free binding assays, one of the components usually comprises or is coupled to a label. A wide variety of labels may be employed-essentially any label that provides for detection of bound protein. The label may provide for direct detection as radioactivity, luminescence, polarisation of light, optical or electron density, etc. or indirect detection such as an epitope tag, an enzyme, etc. The label may be appended to the protein e.g. a phosphate group comprising a radioactive isotope of phosphorous, or incorporated into the protein structure, e.g. a methionine residue comprising a radioactive isotope of sulfur.

A variety of methods may be used to detect a specific label depending on the nature of the label and other assay components. For example, the label may be detected bound to a solid substrate or a portion of the bound complex containing the label may be separated from the solid substrate, and thereafter the label detected. Labels may be directly detected through optical or electron density, radiative emission, nonradiative energy transfer, emission of polarised light, etc., or indirectly detected with antibody conjugates, etc. For example, in the case of radioactive labels, emissions may be detected directly, e.g. with particle counters or indirectly, e.g. with scintillation cocktails and counters.

A difference in the binding affinity of the (poly)peptide of the invention to the target in the absence of the agent as compared with the binding affinity in the presence of the agent indicates that the agent modulates the binding of the Unc-51, ROMA1, and/or 2TM polypeptide to the Unc-51, ROMA1, and/or 2TM binding target. The difference, as used herein, is statistically significant and preferably represents at least a 50%, more preferably at least a 90% difference.

Analogously, in cell-based assays, a difference in Unc-51-dependent activity in the presence and absence of an agent indicates the agent modulates Unc-51, ROMA1, and/or 2TM mediated cellular function or Unc-51, ROMA1, and/or 2TM expression. Such cell-based approaches may involve transient or stable expression assays. In this method, cells are transfected with one or more constructs encoding in sum, a polypeptide comprising a portion of the (poly)peptide of the invention and a reporter under the transcriptional control of an Unc-51, ROMA1, and/or 2TM responsive promotor. The cell may advantageously also be cotransfected with a construct encoding an Unc-51, ROMA1, and/or 2TM activator, e.g. a receptor capable of stimulating Unc-51, ROMA1, and/or 2TM activity, etc. Alternatively, the adipose promotor itself may be linked to a suitable reporter gene, e.g. luciferase, and used in cell-based assays to screen for compounds capable of modulating, via up- or down-regulation, adipose expression.

The methods described herein are particularly suited for automated high-throughput drug screening using robotic liquid dispensing workstations. Similar robotic automation is available for high-throughput cell plating and detection of various assay read-outs.

Candidate agents shown to modulate the expression of the nucleic acid molecules of the invention or association of (poly)peptides of the invention with a binding partner provide valuable reagents to the pharmaceutical industries for animal and human trials. Target therapeutic indications are limited only in that the target Unc-51, ROMA1, and/or 2TM cellular function (e.g. gene expression or association with a binding partner) be subject to modulation. In particular, candidate agents obtained from drug screening assays and the subject compositions, e.g. as Unc-51-derived nucleic acids or therapeutic polypeptides, provide therapeutic applications in diseases associated with body-weight regulation and energy homeostatis, including treatment of obesity, disorders associated with wasting, such as cancer, infectious diseases and HIV infection, or bulimia. As will be discussed herein below, for therapeutic use, the compositions and agents may be administered by any convenient way, preferably parenterally, conveniently in a physiologically acceptable carrier, e.g. phosphate buffered saline, saline, deionized water, or the like. Other additives may be included, such as stabilisers, bactericides, etc. Typically, the compositions are added to a retained physiological fluid such a blood or synovial fluid. Generally, the amount administered will be empirically determined, depending, for example, upon the therapeutic objectives, the route of administration, and the condition of the patient. Typically, the clinician will administer a molecule of the present invention until a dosage is reached that provides the required biological effect. The progress of this therapy is easily monitored by conventional assays.

A method of refining the compound or agent identified by the method of the invention
(a) modeling said compound by peptidomimetics; and
(b) chemically synthesizing the modeled compound.

Peptidomimetics is well known in the art and disclosed, inter alia, in Beeley, Trends Biotech 12 (1994), 213-216, Wiley, Med. Res. Rev. 13 (1993), 327-384, Hruby, Biopolymers 43 (1997), 219-266, or references cited therein or references cited herein above.

Methods of the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Methods for the chemical synthesis and/or the preparation of chemical derivatives and analogues are well known to those skilled in the art and are described in, for example, Beilstein, loc. cit. and “Organic Synthesis”, Wiley, New York, U.S.A., see supra.

It is envisaged in the present invention that the above mentioned peptidomimetics methods and/or methods for chemical synthesis, modification or for refining may also directly be employed on the compounds of the invention, e.g. on the (poly)peptides or on the fusionproteins of the invention.

The present invention relates to a method of producing a composition comprising formulating the compound of the invention, the compound or agents identified by the method(s) described herein or the compound refined by the method(s) described herein above with a pharmaceutically acceptable carrier and/or diluent.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg per dose; however, doses below or above this exemplary range are envisioned, especially considering the aforementioned factors. If the regimen is a continuous infusion, it should also be in the range of 1 μg to 10 mg units per kilogram of body weight per minute, respectively. Progress can be monitored by periodic assessment. The compositions of the invention may be administered locally or systemically. The compositions of the invention may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition.

Additionally, the present invention provides for a method of producing a composition comprising the compound(s) of the invention or the compound(s) or agent(s) identified by the method(s) of the invention comprising the steps of
(a) modifying a compound of the invention, or a compound or agent identified by the method of the invention as a head compound to achieve
(i) modified site of action, spectrum of activity, organ specificity, and/or
(ii) improved potency, and/or
(iii) decreased toxicity (improved therapeutic index), and/or
(iv) decreased side effects, and/or
(v) modified onset of therapeutic action, duration of effect, and/or
(vi) modified pharmakinetic parameters (resorption, distribution, metabolism and excretion), and/or
(vii) modified physico-chemical parameters (solubility, hygroscopicity, color, taste, odor, stability, state), and/or
(viii) improved general specificity, organ/tissue specificity, and/or
(ix) optimized application form and route;
e.g. by
(i) esterification of carboxyl groups, or
(ii) esterification of hydroxyl groups with carbon acids, or
(iii) esterification of hydroxyl groups to, e.g. phosphates, pyrophosphates or sulfates or hemi succinates, or
(iv) formation of pharmaceutically acceptable salts, or
(v) formation of pharmaceutically acceptable complexes, or
(vi) synthesis of pharmacologically active polymers, or
(vii) introduction of hydrophilic moieties, or
(viii) introduction/exchange of substituents on aromates or side chains, change of substituent pattern, or
(ix) modification by introduction of isosteric or bioisosteric moieties, or
(x) synthesis of homologous compounds, or
(xi) introduction of branched side chains, or
(xii) conversion of alkyl substituents to cyclic analogues, or
(xiii) derivatisation of hydroxyl group to ketales, acetates, or
(xiv) N-acetylation to amides, phenylcarbamates, or
(xv) synthesis of Mannich bases, imides, or
(xvi) transformation of ketones or aldehydes to Schiff's bases, oximes, acetales, ketales, enolesters, oxazolines, thiolidines
or combinations thereof; and
(b) formulating the product of said modification with a pharmaceutically acceptable carrier.

Pharmaceutical acceptable carriers are well known in the art, as described herein above. It is envisaged that also the compounds of the invention, i.e. the (poly)peptides or fusionproteins of the invention, the nucleic acid molecules of the invention be employed in the above described method for producing a composition. Preferably, said composition(s) is/are a pharmaceutical composition(s) as described herein.

Therefore, in a more preferred embodiment, the present invention relates to a method of producing a composition comprising the compound(s) of the invention or the compound(s) or agent(s) identified by the method(s) of the invention, wherein said composition is a pharmaceutical composition for preventing or treating obesity, adipositas, eating disorders, bulimia, wasting and/or disorders leading to increased or decreased body weight/body mass, pancreatic dysfunctions (for example diabetes), hypercholesterolemia, dyslipidemia, coronary heart disease, osteoarthritis, gallstones, cancers of the reproductive organs, sleep apnea, disorders related to ROS production, and others, as inter alia, described herein below.

It is particularly preferred that the present invention relates to a method for producing a composition comprising the compound(s) of the invention or compound(s) or agent(s) identified by the method(s) of the invention, wherein said composition is a pharmaceutical composition for preventing, alleviating or treating obesity, adipositas, eating disorders (like bulimia nervosa, anorexia nervosa), wasting syndromes (like cachexia), mitochondrial disorders, pancreatic dysfunctions (like diabetes), the prevention of insulin resistance, disorders related to ROS production (like response to infections, cancer, aging). A number of diseases and disorders are thought to be caused by or be associated with alterations in mitochondrial metabolism and/or inappropriate induction or suppression of mitochondria-related functions leading to apoptosis. These include, by way of example and not limitation, chronic neurodegenerative disorders such as Alzheimer's disease (AD) and Parkinson's disease (PD); auto-immune diseases; diabetes mellitus, including Type I and Type II; mitochondria associated diseases, including but not limited to congenital muscular dystrophy with mitochondrial structural abnormalities, fatal infantile myopathy with severe mtDNA depletion and benign “later-onset” myopathy with moderate reduction in mtDNA, MELAS (mitochondrial encephalopathy, lactic acidosis, and stroke) and MIDD (mitochondrial diabetes and deafness); MERFF (myoclonic epilepsy ragged red fiber syndrome); arthritis; NARP (Neuropathy; Ataxia; Retinitis Pigmentosa); MNGIE (Myopathy and external ophthalmoplegia; Neuropathy; Gastro-Intestinal; Encephalopathy), LHON (leber's; Hereditary; Optic; Neuropathy), Kearns-Sayre disease; Pearson's Syndrome; PEO (Progressive External Ophthalmoplegia); Wolfram syndrome DIDMOAD (Diabetes Insipidus, Diabetes Mellitus, Optic Atrophy, Deafness); Leigh's Syndrome; dystonia; schizophrenia; and hyperproliferative disorders, such as cancer, tumors and psoriasis.

In yet another embodiment, the present invention provides for a composition comprising
(a) an inhibitor of the (poly)peptide of the invention or identified by the method or refined by the method of the invention;
(b) an inhibitor of the expression of the gene identified by the method described herein or the nucleic acid molecule of the invention; and/or
(c) a compound identified by the method of the invention.

Said inhibitor of the (poly)peptide of the invention may be a compound which functions as inhibitor of the wildtype (poly)peptide of the invention, the Unc-51, ROMA1, and/or 2TM protein. Said inhibitor may lead to induction of weight loss may influence regulatory cells (pancreatic beta-cells) and thereby improve beta-cell function or preventing insulin resistance, it may change ROS (reactive oxygen species) production leading to a decreased ROS concentration (causing reduced molecular damage in aging, cancerogenesis and increased ischemic tolerance). However opposite effects could occur due to tissue specific reactions and metabolic situation. Said inhibitor may also be an inhibitor specifically interacting with (a) mutated form(s) of the (poly)peptide of the invention and thereby lead to a decrease in body weight/body mass or to an maintainance of the current body weight/body mass.

It is to be understood that the term “inhibitor” of the (poly)peptide identified by the methods of the invention also relates to (an) inhibitor(s) which influence the activity and/or function of interacting (poly)peptides as identified by the method of the present invention. Said interaction may be direct or indirect. Said “inhibitor” may also interfere with and/or modify the interaction of the (poly)peptide of the invention with its binding targets/agents as defined herein. The above described applies mutatis mutandis for the term “inhibitor of the expression of the nucleic acid molecule of the invention or of the gene identified by the method(s) of the invention”. Said inhibitor may interfere with transcriptional and/or translational processes.

Similarly, the present invention relates to a composition comprising
(a) a stimulator of the (poly)peptide of the invention or of the (poly)peptide identified or refined by the method(s) described herein above;
(b) a stimulator of the expression of the nucleic acid molecule of the invention or of the gene identified by the method(s) of the invention;
(c) a compound identified by the method(s) of the invention; and/or
(d) the vector of the invention.

The term “stimulation of the (poly)peptide” of the invention relates to a compound which functions as a stimulator (activator) of the (poly)peptides of the invention. Said stimulator/activator may lead to a induction of weight gain and may be useful for the treatment of wasting. It may also change the ROS production which may lead to increased efficacy in (a) immune response(s). Yet, opposite effects are also envisaged, due to tissue specific reactions and metabolic situations. The here described “stimulators” may, inter alia, lead to an increased interaction of the (poly)peptide of the invention with its binding target. The term also relates to a stimulator/activator of the mutated form(s) of the (poly)peptides of the present invention. Said stimulator(s) of the mutated form(s) may lead to an increase in body weight/body mass or to an maintenance of the current body weight/body mass.

“Inhibitors” as well as “stimulators of the (poly)peptide of the invention” may be deduced and/or evaluated by methods known in the art and disclosed herein.

The term “stimulator of a (poly)peptide identified or refined by the method(s) of the present invention” relates also to a stimulator which influences the activity/function of (interacting) (poly)peptides as identified by the method of the present invention they may interact with said (poly)peptides in either direct or indirect fashion. As already mentioned for the term “inhibitor” as defined herein above, the above said applies, mutatis mutantis, for the term “stimulator of the expression of the nucleic acid molecule of the invention or of the gene identified by the method(s) of the invention”.

The above mentioned “inhibitors” and “stimulators” not only relate to (poly)peptides, but may also comprise small molecules which bind to, interfere with and/or interact with the (poly)peptides and/or nucleic acid molecules of the invention or with (poly)peptides and/or genes identified by the method(s) of the invention. Examples of such small molecules comprise, but are not limited to small peptides, anorganic and/or organic substances or peptide-like molecules, like peptide-analogs comprising D-amino acids. Said “inhibitors” and “stimulators” may further comprise antibodies, derivatives and/or fragments thereof, aptamers or specific (oligo)nucleotides. The “inhibitors” and “stimulators” may also be part of the pharmaceutical and/or diagnostic compositions as disclosed herein.

As pointed out herein above, said “inhibitors” or “stimulators” may also comprise small organic compounds as defined herein above.

In addition, the present invention relates to a composition comprising a nucleic acid molecule of the invention, a (poly)peptide of the invention, a fusion protein of the invention, an antibody or (a) fragment(s) or derivative(s) thereof or an aptamer of the invention or an anti-sense oligonucleotide of the invention. Furthermore, said composition may comprise (poly)peptides, nucleic acid molecules, genes and/or compounds or agents as identified by the methods of the present invention.

In a preferred embodiment of the invention, said composition is a pharmaceutical composition. Pharmaceutical compositions comprising, optionally, pharmaceutically acceptable carriers have been described herein above. The pharmaceutical compositions of the present invention are particularly useful for the treatment and/or the prevention of complex disorders of appetite regulation and/or energy metabolism. It is particularly preferred, but not limited to, that said pharmaceutical composition is employed in treating and/or preventing obesity, adipositas, eating disorders, bulimia, disorders of body weight/body mass, pancreatic dysfunctions (for example diabetes), disorders related to ROS production, and others. It is, however, also envisaged that said pharmaceutical compositions be used in disorders like, inter alia, wasting (cachexia), weight loss due to cancer or infectious diseases or weight loss in immuno-compromised patients, like, HIV-patients.

It is furthermore envisaged that the pharmaceutical composition of the invention may be used in combination with other agents employed in the treatment of body weight/mass disorders. Said agents may comprise, but are not limited to, agents reducing/enhancing food intake, agents blocking/activating nutrient absorption, agents increasing/decreasing thermogenesis, agents modulating fat and/or protein metabolism or storage, agents modulating the central contoller regulating body weight. Said agents may, inter alia, comprise, agents like sibutramine, orlistat, ephedrine or caffeine, diethylpropione, phentermine, fluoxetine, sertraline, or phenylpropanolamine.

Furthermore, the present invention relates to a composition comprising a nucleic acid molecule of the invention, a (poly)peptide of the invention, a fusionprotein of the invention, an antibody, a derivative or fragment thereof, an aptamer of the invention at least a primer or a set of primers as defined herein or an anti-sense oligonucleotide of the invention. Particularly preferred primers are primers as employed in the appended examples. It is, e.g., envisaged that primers deduced from the nucleic acid molecules of the invention are employed for diagnostic or scientific purposes. Said primers may, inter alia, be employed to find and/or verify mutations of Unc-51, ROMA1, and/or 2TM genes in individuals. Preferably, said individuals are humans. Furthermore, primers as deduced from the nucleic acid sequences disclosed herein, may be employed/used to detect and/or isolate homologous sequences in further species.

In a particular preferred embodiment said composition is a diagnostic composition. Said diagnostic composition may comprise the components as defined herein above wherein said components are bound to/attached to and/or linked to a solid support as defined herein above. It is furthermore envisaged, that said diagnostic composition comprises a compound(s) of this invention on (micro-)chips. Therefore, said diagnostic composition may, inter alia, comprise the nucleic acid molecules of the invention on so-called “gene chips” or the (poly)peptides of the invention on so-called “protein-chips”. Diagnostic gene chips may comprise a collection of the nucleic acid molecules of the invention that, e.g., specifically detect mutations in the Unc-51-gene of animals, in particular of humans. Said diagnostic compositions and in particular the diagnostic gene chip as described herein above may be particularly useful for screening patients for (genetic) defects underlying, e.g. obesity, adipositase, disorders of body weight/body mass, pancreatic dysfunctions (for example diabetes), disorders related to ROS production, and others.

It is preferred that said compounds of the present invention to be employed in a diagnostic composition are detectably labeled. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds.

Furthermore, the present invention relates to the use of
(a) an inhibitor of the (poly)peptide identified or refined by the method of the invention;
(b) an inhibitor of the expression of the gene identified by the method of the invention; and/or
(c) a compound identified by the method of the invention;
   for the preparation of a pharmaceutical composition for the treatment of obesity, adipositas, eating disorders, wasting syndromes (e.g. cachexia), mitochondrial disorders, pancreatic dysfunctions (for example diabetes), disorders related to ROS production, and others.

Similarly, the present invention also provides for the use of
(a) a stimulator of the (poly)peptide identified or refined by the method of the invention;
(b) a stimulator of the expression of the gene identified by the method of the invention; and/or
(c) a compound identified by the method of the invention;
   for the preparation of a pharmaceutical composition for the treatment of obesity, adipositas, eating disorders, wasting syndromes (cachexia), mitochondrial disorders, pancreatic dysfunctions (for example diabetes), disorders related to ROS production, and others.

Furthermore, the present invention relates to the use of an agent as identified by the method of the invention for the preparation of a pharmaceutical composition for the treatment, alleviation and/or prevention of obesity, adipositas, eating disorders, wasting syndromes (cachexia, also in cancer, HIV-infections), mitochondrial disorders as described herein, pancreatic dysfunctions (for example diabetes) (like diabetes), disorders related to ROS production (like cancer, aging, infections), and others.

In a particular preferred embodiment, the present invention relates to the use of a fruit fly as defined in herein above for the detection of polypeptides capable of contributing to membrane stability and/or function in organelles, capable of modifying mitochondrial proteins, and/or capable of influencing cellular metabolism.

Furthermore, the invention provides for a kit comprising at least one of a nucleic acid molecule, a vector, a host, a polypeptide, a fusion protein, an antibody or a fragment or derivative thereof or an antiserum, an aptamer or another receptor and an anti-sense oligonucleotide of the invention. Advantageously, the kit of the present invention further comprises, optionally (a) reaction buffer(s), storage solutions and/or remaining reagents or materials required for the conduct of scientific or diagnostic assays or the like. Furthermore, parts of the kit of the invention can be packaged individually in vials or bottles or in combination in containers or multicontainer units.

The kit of the present invention may be advantageously used, inter alia, for carrying out the method of producing a (poly)peptide of the invention and could be employed in a variety of applications referred herein, e.g., as diagnostic kits, as research tools or vaccination tools. Additionally, the kit of the invention may contain means for detection suitable for scientific medical and/or diagnostic purposes. The manufacture of the kits follows preferably standard procedures which are known to the person skilled in the art.

### BRIEF DESCRIPTION OF THE DRAWINGS

FIG. 1. Nucleotide and amino acid sequences of the Drosophila Uncoupling protein (UCPy)

FIG. 1A: Full length cDNA of the Drosophila Uncoupling protein (UCPy) (SEQ ID NO: 1)

FIG. 1B: Deduced open reading frame of the Drosophila Uncoupling protein (UCPy) (SEQ ID NO: 2)

FIG. 1C. Amino acid sequence (one letter code) encoding the Drosophila Uncoupling protein (UCPy) (SEQ ID NO:3)

FIG. 2A-E. CLUSTAL X (1.8) multiple amino acid sequence alignment of Unc-51-like protein from Drosophila melanogaster, mouse, and human. The alignment was produced using the multisequence alignment program of Clustal V software (Higgins, D. G. and Sharp, P. M. (1989). CABIOS, vol. 5, no. 2, 151-153. hsNP_{—}003556 is the human ULK-1 protein, mmNP_{—}033495 refers to mouse ULK-1, hsNP_{—}055498 is the human ULK-2 protein, mmBAA77341 refers to mouse ULK-2, dmAAF49878 refers to the Drosophila Unc-51 like protein. Identical amino acid residues are marked with a star.

FIG. 3. Expression of Unc51 in mammalian tissues.

FIG. 3A: Real-time PCR analysis of unc51-like kinase 1 in wildtype mouse tissues. The relative RNA-expression is shown on the left hand side, the tissues tested are given on the horizontal line. WAT=white adipose tissue, BAT=brown adipose tissue

FIG. 3B: Real-time PCR mediated comparison of unc51-like kinase 1 expression during the differentiation of 3T3-L1 cells from preadipocytes to mature adipocytes. The relative RNA-expression is shown on the left hand side, the days of differention are shown on the horizontal line (d0=day 0, start of the experiment, until d10=day 10)

FIG. 3C: Real-time PCR mediated comparison of unc51-like kinase 1 expression during the differentiation of 3T3-F442A from preadipocytes to mature adipocytes. The relative RNA-expression is shown on the left hand side, the days of differention are shown on the horizontal line (d0=day 0, start of the experiment, until d10=day 10)

FIG. 4. Nucleotide and protein sequences encoding Roma1

FIG. 4A. Nucleotide sequence of the open reading frame encoding the Drosophila melanogaster Roma1 gene (GADFLY Accession Number CG15081, pp-CT34956) (SEQ ID NO: 4)

FIG. 4B. Deduced amino acid sequence (shown in the one-letter-code) of the Drosophila melanogaster Roma1 protein (GADFLY Accession Number CG15081, pp-CT34956) (SEQ ID NO: 5)

FIG. 5. shows the the amino acid sequence alignments among the ROMA1 protein (line 1; SEQ ID NO:5), human BAP37 protein (line 2, Genbank Accession Number XP_{—}006639.1), mouse BAP37 protein (line 3, Genbank Accession Number NP_{—}031557.1). The alignment was produced using the multisequence alignment program of Clustal V software (Higgins, D. G. and Sharp, P. M. (1989). CABIOS, vol. 5, no. 2, 151-153.)

FIG. 6. Expression of ROMA1 in mammalian tissues.

FIG. 6A: Real-time PCR analysis of ROMA expression in wildtype mouse tissues. The relative RNA-expression is shown on the left hand side, the tissues tested are given on the horizontal line. WAT=white adipose tissue, BAT=brown adipose tissue

FIG. 6B: Real-time PCR mediated comparison of ROMA expression during the differentiation of 3T3-L1 cells from preadipocytes to adipocytes. The relative RNA-expression is shown on the left hand side, the days of differention are shown on the horizontal line (d0=day 0, start of the experiment, until d10=day 10)

FIG. 7A-D shows the amino acid sequences (one-letter code) of human 2TM homologous proteins

FIG. 8 shows the amino acid sequence alignments among the 2TM proteins from Drosophila melanogaste (GadFly accession number CG7620), mouse (GenBank accession number BAB26124), and human (Accession numbers ENSP00000242518-SEQ ID NO:7, BG432914-SEQ ID NO:6, ENSP00000243785-SEQ ID NO:8, and ENSP00000250594-SEQ ID NO:9). The alignment was produced using the multisequence alignment program of Clustal V software (Higgins, D. G. and Sharp, P. M. (1989). CABIOS, vol. 5, no. 2, 151-153.)

FIG. 9 shows a transmembrane domain plot of Drosophila (FIG. 9A) and human (FIG. 9B; SEQ ID NO:7) 2TM proteins. Calculated following: J. Glasgow et al., Proc. Sixth Int. Conf. on Intelligent Systems for Molecular Biology. 175-182, AAAI Press, 1998.

FIG. 10 shows the mitochondrial localisation of tagged 2TM protein in transfected mammalian NIH3T3 cells. NIH3T3 cells were transiently transfected with an expression vector for mouse 2TM protein, specifically labeled with a FLAG-tag, fixed, and immunostained with an antisera against the FLAG-tag (see Examples).

### EXAMPLES

A better understanding of the present invention and of its many advantages will be had from the following examples, given by way of illustration.

### Example 1

### Cloning of a Drosophila melanogaster Gene with Homology to Human Uncoupling Proteins (UCPs)

A BLAST homology search was performed in a public database (NCBI/NIH) looking for Drosophila genes with sequence homology to the human UCP2 and UCP3 genes. The search yielded sequence fragments of a family of Drosophila genes with UCP homology. They are clearly different to the next related mitochondrial proteins (oxoglutarate carrier).

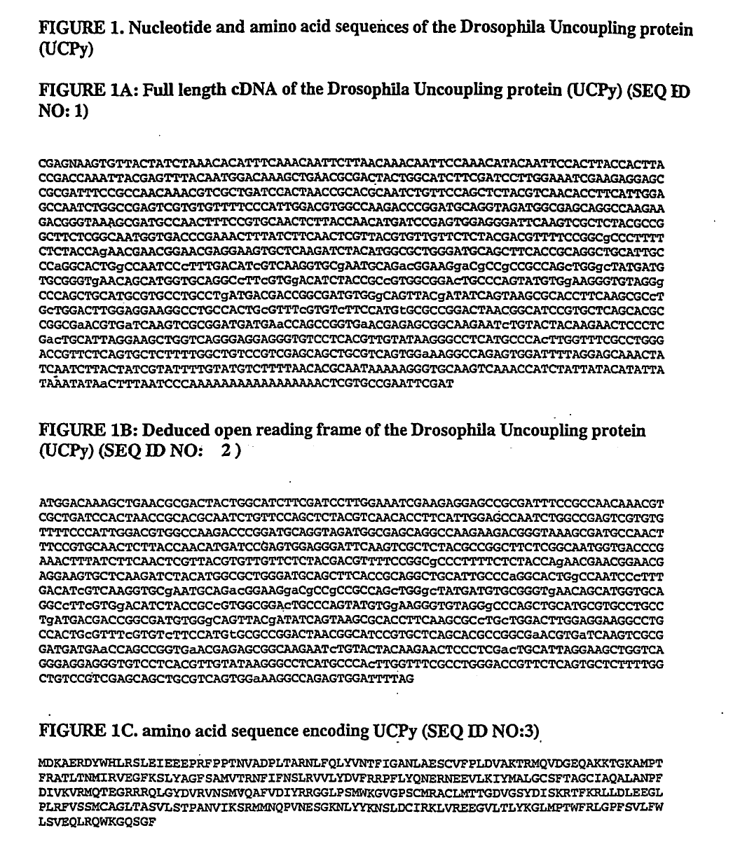

Using the sequence fragment of one of this genes (here called dUCPy), a PCR primer pair was generated (Upper 5-CTAAACAAACAATTCCAAACATAG (SEQ ID NO: 10), Lower 5′-AAAAGACATAGAAAATACGATAGT (SEQ ID NO: 11) and a PCR reaction performed on Drosophila cDNA using standard PCR conditions. The amplification product was radioactively labelled and used to screen a cDNA library made from adult Drosophila flies (Stratagene). A full-length cDNA clone was isolated, sequenced, and used for further experiments. The nucleotide sequence and the deduced open reading frame of UCPy is shown in FIGS. 1A and B.

### Example 2

### Cloning of the dUCPy cDNA into an Drosophila Expression Vector

In order to test the effects of dUCPy expression in Drosophila cells, dUCPy cDNA was cloned into the expression vector pUAST (Ref.: Brand A & Perrimon N, Development 1993, 118:401-415) using the restriction sites NotI and KpnI. The resulting expression construct was injected into the germline of Drosophila embryos and Drosophila strains with a stable integration of the construct were generated. Since the expression vector pUAST is activated by the yeast transcription factor Gal4 which is normally absent from Drosophila cells dUCPy is not yet expressed in these transgenic animals. If pUAST-dUCPy flies are crossed with a second Drosophila strain that expresses Gal4 in a tissue specific manner the offspring flies of this mating will express dUCPy in the Gal4 expressing tissue.

The cross of pUAST-dUCPy flies with a strain that expresses Gal4 in all cells of the body (under control of the actin promoter) showed no viable offspring. This means that dUCPy overexpression in all body cells is lethal. This finding is consistent with the assumption that dUCPy overexpression could lead to a collapse of the cellular energy production.

Expression of dUCPy in a non-vital organ like the eye (Gal4 under control of the eye-specific promoter of the “eyeless” gene) results in flies with visibly damaged eyes. This easily visible eye phenotype is the basis of a genetic screen for gene products that can modify UCP activity.

### Example 3

### dUCPy Modifier Screen

Parts of the genomes of the strain with Gal4 expression in the eye and the strain carrying the pUAST-dUCPy construct were combined on one chromosome using genomic recombination. The resulting fly strain has eyes that are permanently damaged by dUCPy expression. Flies of this strain were crossed with flies of a large collection of mutagenized fly strains. In this mutant collection a special expression system (EP-element, Ref.: Rørth P, Proc Natl Acad Sci USA 1996, 93(22):12418-22) is integrated randomly in different genomic loci. The yeast transcription factor Gal4 can bind to the EP-element and activate the transcription of endogenous genes close the integration site of the EP-element. The activation of the genes therefore occurs in the same cells (eye) that overexpress dUCPy. Since the mutant collection contains several thousand strains with different integration sites of the EP-element it is possible to test a large number of genes whether their expression interacts with dUCPy activity. In case a gene acts as an enhancer of UCP activity the eye defect will be worsened; a suppressor will ameliorate the defect.

Using this screen genes with suppressing activity were discovered that are here called ROMA1 and/or 2TM, a gene that is enhancing the eye-phenotype is Unc-51.

### Example 4

### Cloning of Unc-51, ROMA1, and/or 2TM from Drosophila

Genomic DNA neighbouring to the eye-defect modifying EP-element was cloned by inverse PCR and sequenced. This sequence was used for a BLAST search in a public Drosophila gene database.

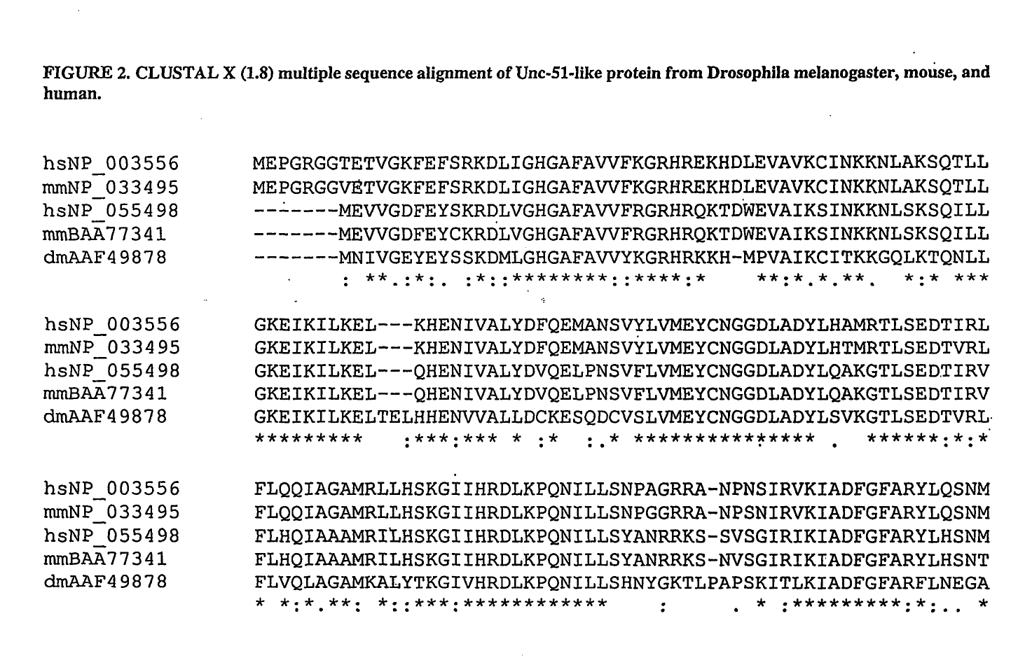

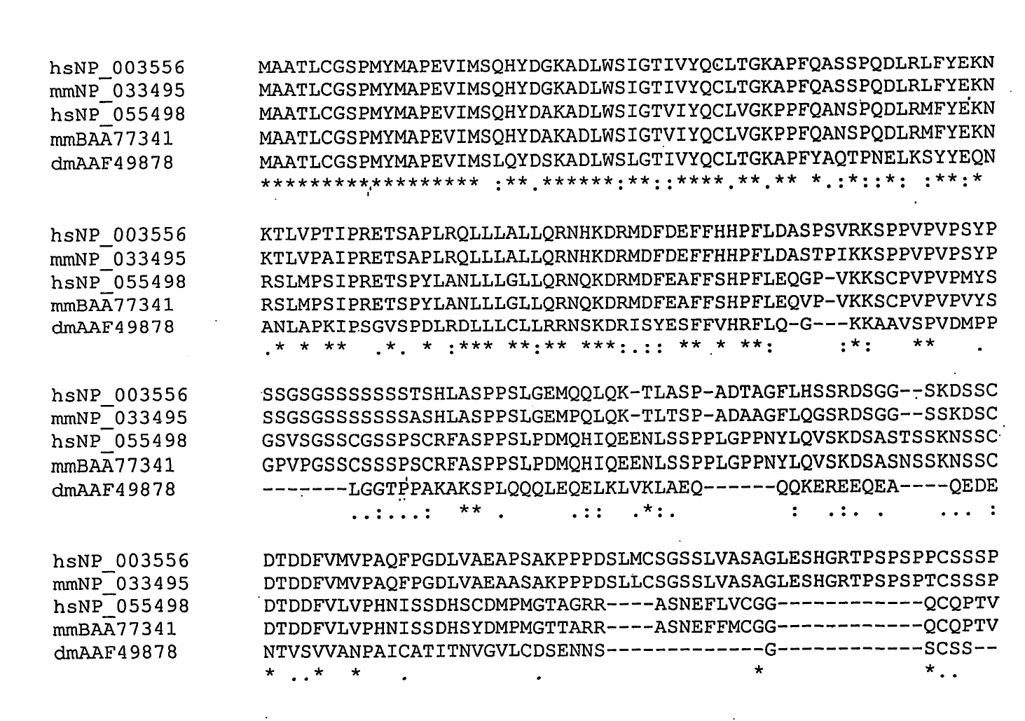

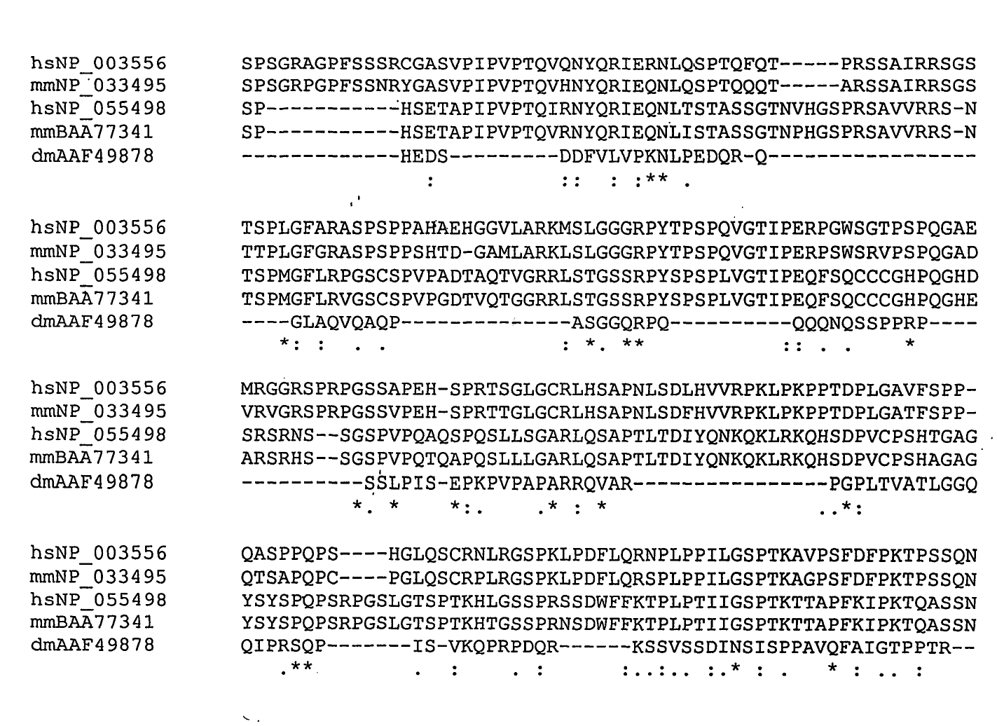

Unc-51: The database search indicated that the EP-element is integrated upstream of the ATG of a predicted transcript annotated as CG10967 (Drosophila Genome Project). The deduced protein sequence of CG10967 is shown in FIG. 2.

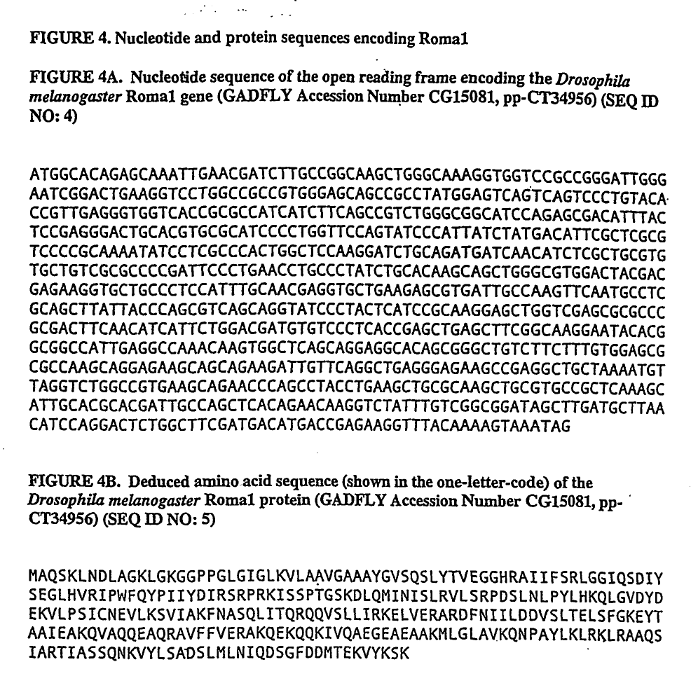

ROMA1: The database search indicated that the EP-elementis integrated 246 bp upstream of the ATG of a predicted transcript annotated as CG15081, pp-CT34956 (Drosophila Genome Project). The nucleotide sequence of CG15081, pp-CT34956 is shown in FIG. 4A. The deduced protein sequence is shown in FIG. 4B.

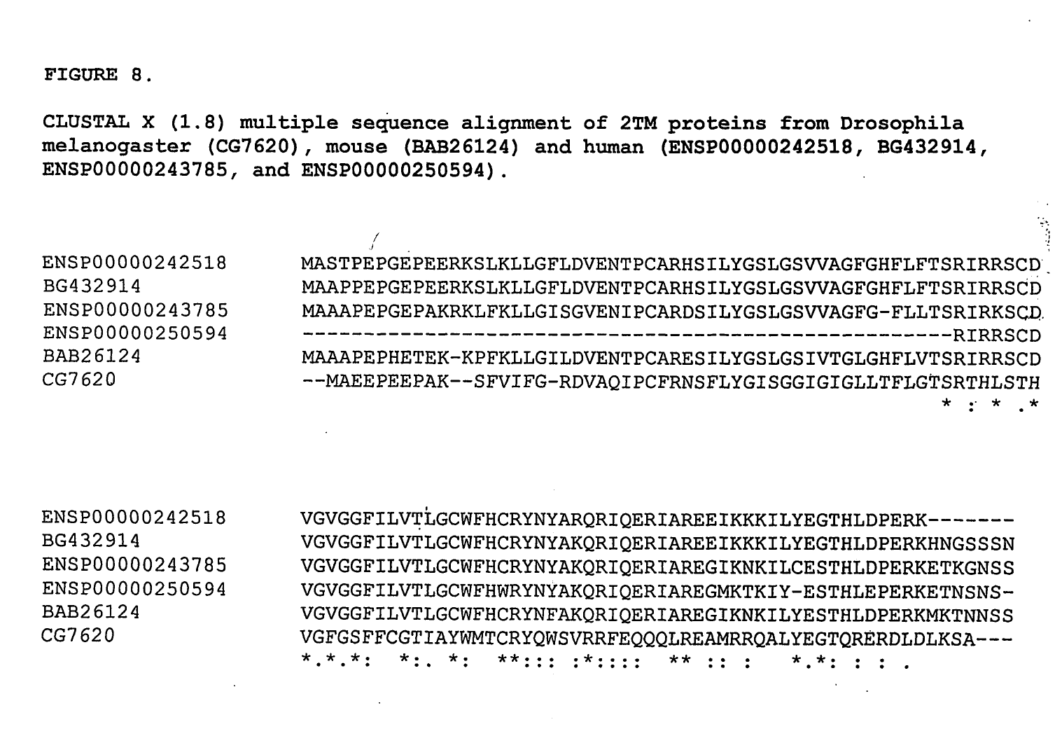

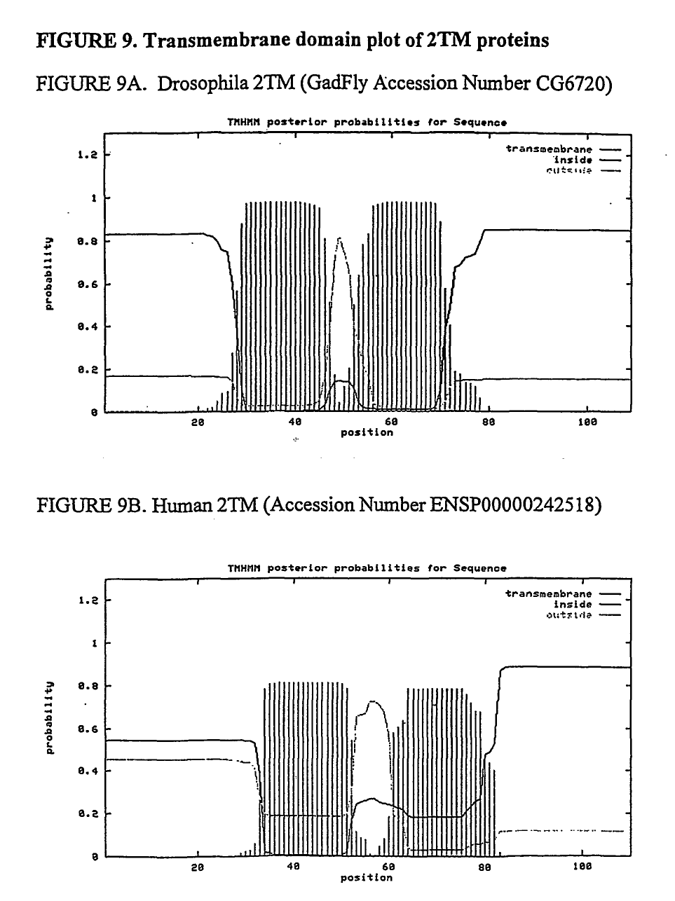

2TM: The database search indicated that the EP-element is integrated in the second exon of a predicted transcript annotated as CG7620 (Drosophila Genome Project); due to the integration site, the gene CG7620 is most likely inactivated (‘knock-out’). The deduced protein sequence of CG7620 is shown in FIG. 8.

### Example 5

### Sequence Analysis of the Proteins of the Invention

Using the sequences of the Drosophila genes, a BLAST search for mammalian homologues was performed in public databases (for example, National Center for Biotechnology Information (NCBI) at the National Institutes of Health (NIH)). The similiarties are shown in Table 1, supra. The sequences of Drosophila, human, and mouse homologues of Unc-51 is shown as multiple sequence alignment in FIG. 2. The alignment was produced using the multisequence alignment program of Clustal V software (Higgins & Sharp, 1989, CABIOS 5, 151-153.)

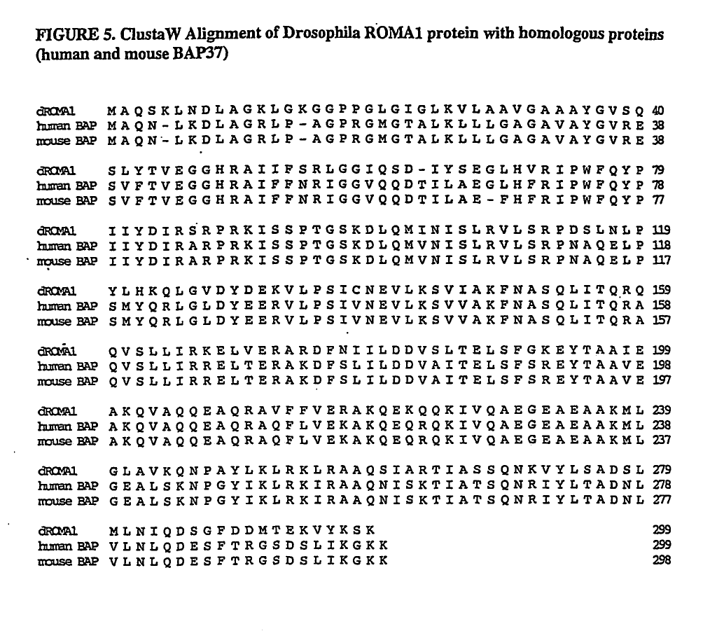

The human homologue of ROMA1 is annotated as “B-cell associated protein” under accession number XP_{—}006639.1 (Identities=189/261 (72%), Positives=236/261 (90%); the DNA sequence is also disclosed as AX026549 in patent application WO 00/40752), the mouse homologue is available under accession number NP_{—}031557.1 (Identities=185/260 (71%), Positives=231/260 (88%)). The nucleotide and protein sequences of ROMA1 are shown in FIG. 4A and FIG. 4B, respectively, and the protein sequence alignment of Drosophila ROMA1 with human and mouse homologous proteins is shown in FIG. 5. The alignment was produced using the multisequence alignment program of Clustal V software (Higgins & Sharp, 1989, CABIOS 5, 151-153.)

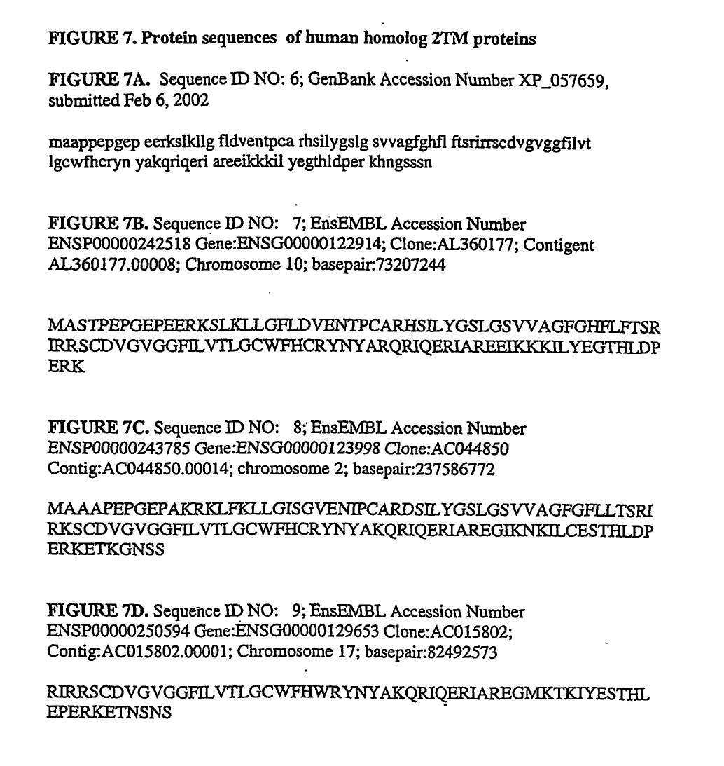

Protein sequences of human homolog 2TM proteins is shown in FIG. 7 A to D, the similiarties are shown in Table 2, supra, and the sequence alignment of the protein sequences of Drosophila 2TM protein with human and mouse homologous proteins is shown in FIG. 8. The 2TM proteins shown two characteristic transmembrane domains, see FIG. 8.

### Example 6

### Expression of the Polypeptides in Mammalian Tissues

For analyzing the expression of the polypeptides disclosed in this invention in mammalian tissues, several mouse strains (preferrably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferrably 22° C.), 40 percent humidity and a light/dark cycle of preferrably 14/10 hours. The mice were fed a standard chow (for example, from ssniff Spezialitäten GmbH, order number ssniff M-Z V1126-000). Animals were sacrificed at an age of 6 to 8 weeks. The animal tissues were isolated according to standard procedures known to those skilled in the art, snap frozen in liquid nitrogen and stored at −80° C. until needed.

For analyzing the role of the proteins disclosed in this invention in the in vitro differentiation of different mammalian cell culture cells for the conversion of pre-adipocytes to adipocytes, mammalian fibroblast (3T3-L1) cells (e.g., Green & Kehinde, Cell 1: 113-116, 1974) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu. et al., J. Biol. Chem. 276:11988-95, 2001; Slieker et al., BBRC 251: 225-9, 1998). At various time points of the differentiation procedure, beginning with day 0 (day of confluence) and day 2 (hormone addition; for example, dexamethason and 3-isobutyl-1-methylxanthin), up to 10 days of differentiation, suitable aliquots of cells were taken every two days. Alternatively, mammalian fibroblast 3T3-F442A cells (e.g., Green & Kehinde, Cell 7: 105-113, 1976) were obtained from the Harvard Medical School, Department of Cell Biology (Boston, Mass., USA). 3T3-F442A cells were maintained as fibroblasts and differentiated into adipocytes as described previously (Djian, P. et al., J. Cell. Physiol., 124:554-556, 1985). At various time points of the differentiation procedure, beginning with day 0 (day of confluence and hormone addition, for example, Insulin), up to 10 days of differentiation, suitable aliquots of cells were taken every two days. 3T3-F442A cells are differentiating in vitro already in the confluent stage after hormone (insulin) addition.

RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferrably using Superscript II RNaseH⁻ Reverse Transcriptase, from Invitrogen, Karlsruhe, Germany) and subjected to Taqman analysis preferrably using the Taqman 2×PCR Master Mix (from Applied Biosystems, Weiterstadt, Germany; the Mix contains according to the Manufacturer for example AmpliTaq Gold DNA Polymerase, AmpErase. UNG, dNTPs with dUTP, passive reference Rox and optimized buffer components) on a GeneAmp 5700 Sequence Detection System (from Applied Biosystems, Weiterstadt, Germany).

Taqman analysis was performed preferrably using the following primer/probe pair:

For the Amplification of unc51:

Mouse unc51-like kinase 1 forward primer (SEQ ID NO: 12): 5′-CCA TGC TGT GCA AAT GGT ACA-3′; mouse unc51-like kinase 1 reverse primer (SEQ ID NO: 13): 5′-TCG GTA CAC AGC CCT CTC G-3′; Taqman probe (SEQ ID NO: 14): (5/6-FAM) TCA GCT GCC CTT GAT GAG ATG TTC CAG (5/6-TAMRA)

For the Amplification of ROMA1:

Mouse ROMA forward primer (SEQ ID NO: 15): 5′-CAC CAC CAG AGA AGT TGG CA-3′; mouse ROMA reverse primer (SEQ ID NO: 16): 5′-GGC TGT GCT TGA CCC CG-3′; Taqman probe (SEQ ID NO: 17): (5/6-FAM) CTT GTC CAG CTT GGA GGA GCC AGC (5/6-TAMRA)

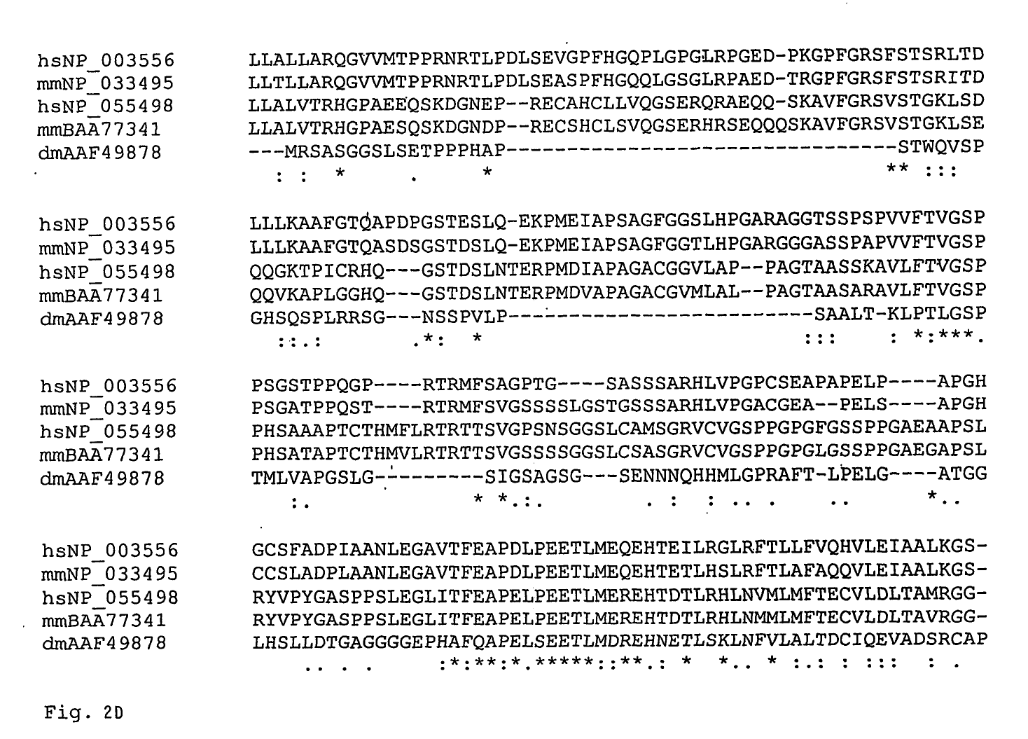

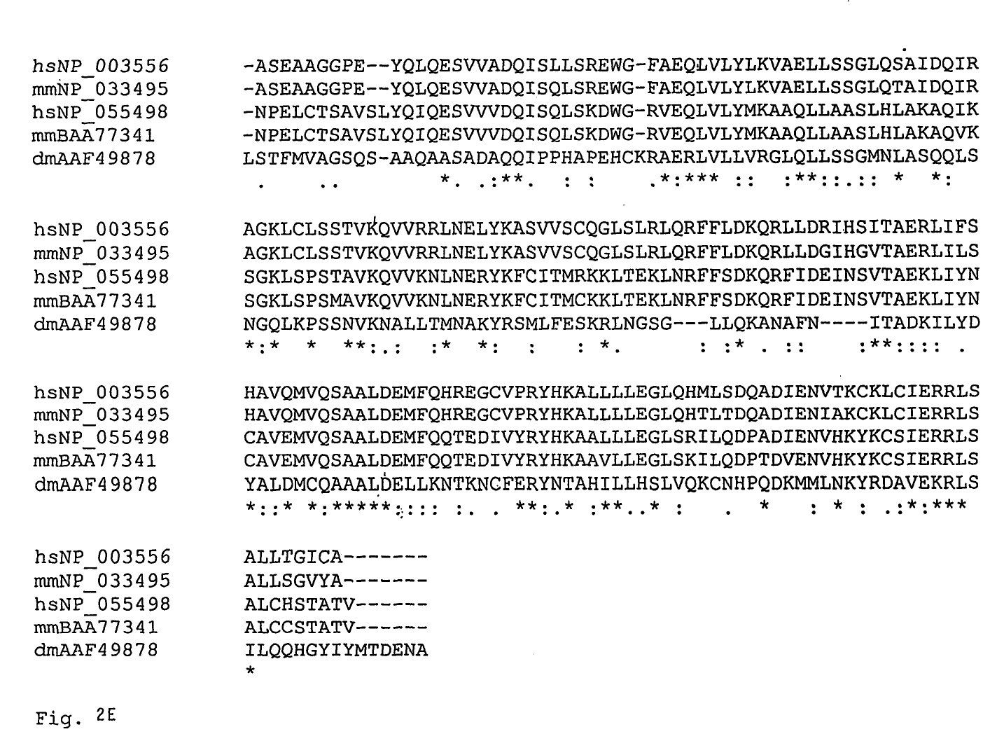

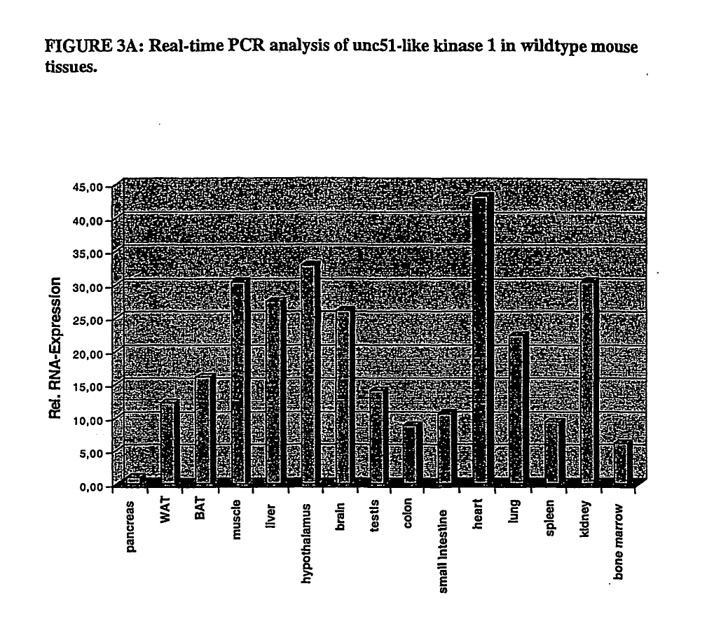

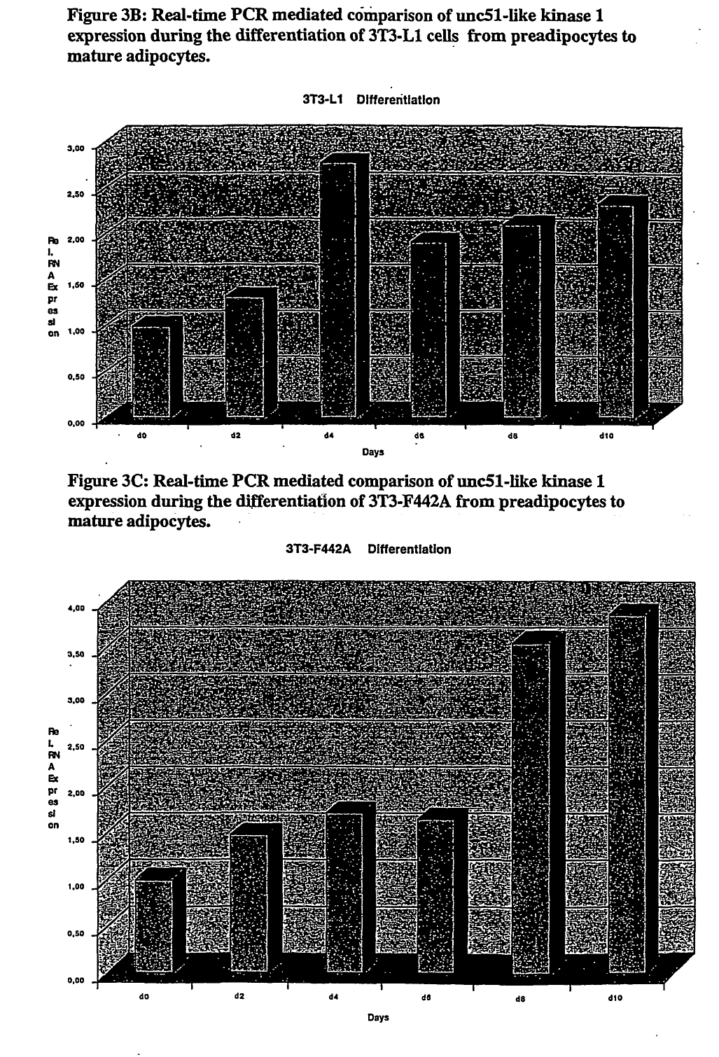

As shown in FIG. 3A, real time PCR (Taqman) analysis of the expression of unc51-like protein in mammalian (mouse) tissues revealed revealed that unc51-like kinase 1 is ubiquitously expressed in different mammalian tissues, showing higher levels of expression in heart, hypothalamus, muscle, kidney, liver, brain, and lung tissues. A clear expression in white adipocyte tissue (WAT) and brown adipocyte tissue (BAT) is seen, confirming a role in the regulation of energy homeostasis and thermogenesis. The unc51-like protein was also examined in the in vitro differentiation models for the conversion of pre-adipocytes to adipocytes, as described above. As shown in FIG. 3, unc51-like protein shows a 2- to 3-fold induction of its expression during differentiation, starting on day 4 of differentiation in 3T3-L1 cells (FIG. 3B) and on day 8 of differentiation in 3T3-F442A cells (FIG. 3C).

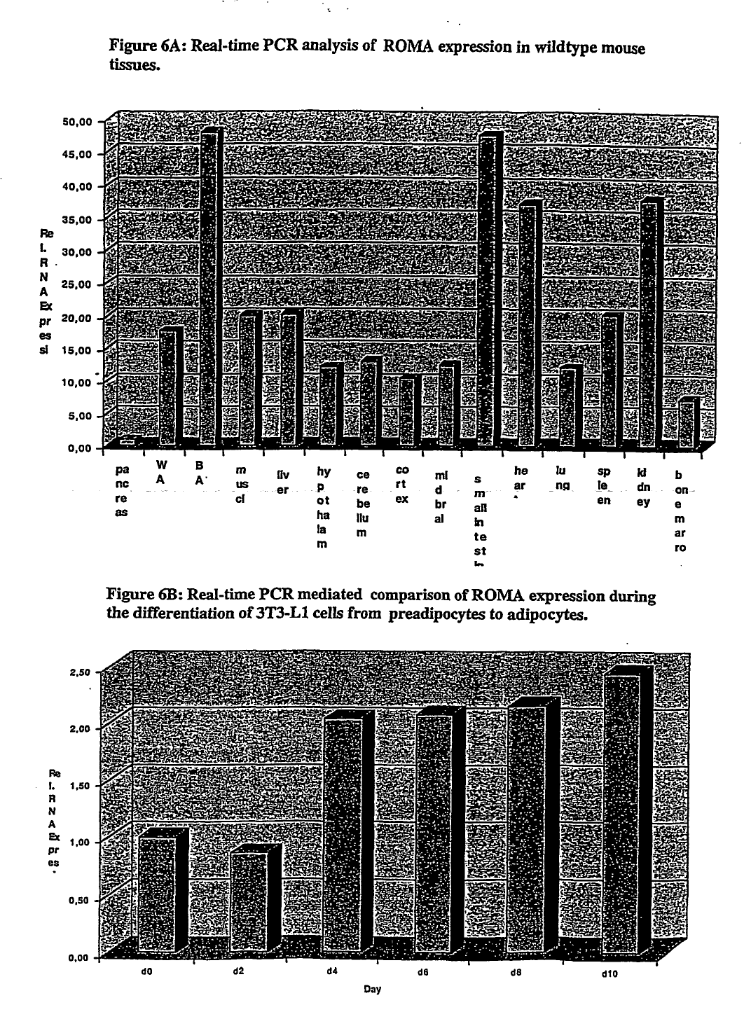

As shown in FIG. 6A, real time PCR (Taqman) analysis of the expression of ROMA1 protein in mammalian (mouse) tissues revealed revealed that ROMA1 is ubiquitously expressed in various mammalian tissues, showing the highest levels of expression in brown adipocyte tissue (BAT), small intestine, heart, and kidney tissues. A clear expression in white adipocyte tissue (WAT) is also observed, confirming a role in the regulation of energy homeostasis and thermogenesis. The ROMA1 protein was also examined in the in vitro differentiation models for the conversion of pre-adipocytes to adipocytes, as described above. As shown in FIG. 6B, expression of ROMA1 is clearly increased during the in vitro differentiation of 3T3-L1 cells from pre-adipocytes to mature adipocytes (starting on day 4).

### Example 7

### Subcellular Localisation of the Mammalian 2TM Protein

Mammalian cells were transiently transfected with an expression vector for mouse 2TM protein, fixed and immunostained with an suitable antisera and analyzed. To determine the subcellular localisation of the mammalian 2TM protein, NIH3T3 cells were transfected with preferrably FLAG- and HA-tagged 2TM proteins which where cloned into a suitable expression vector (for example, pcDN3.1; from InVitrogen; standard vector for eukaryotic expression with CMV-promoter). The Flag-Tag was introduced in the reading frame of the 2TM protein by PCR mediated mutagenesis using the primers 2TMFLAG.up (SEQ ID NO: 18; 5′-AGA AAG CTT GTG CCC ATG GCG GCC GCC C-3′) and 2TMFLAG.low (SEQ ID NO: 19; 5′ TAT CGA ATT CCT ACT TGT CAT CAT CGT CCT TGT AGT CGC TGC TGT TGT TGG TCT TC-3′). The primer with SEQ ID NO:18 introduces a specific endonuclease restriction side (for example, HindIII) 6 base pairs upstream of the start codon (ATG). The primer with SEQ ID NO:19 introduces the Flag-Tag in frame at the 3′-prime end of the open reading frame of the protein and a specific endonuclease restriction side (for example, EcoRI) 6 base pairs downstream of the stop codon.

The polymerase chain reaction (PCR) was performed on cDNA obtained from mammalian (for example, mouse) skeletal muscle using preferrably the “High Fidelity Platinum Taq polymerase” (preferrably from Invitrogen, Karlsruhe, Germany); any other Taq polymerase could also be used and standard PCT techniques as known to those skilled in the art could be employed. cDNA synthesis was performed using 6 RNA, 1 liter oligo dT primers (preferably at a concentration of 500 gram/ml), and the Superscript II Kit (from Invitrogen, Karlsruhe, Germany) and used according to the suppliers protocol. Following the endonuclease restriction with preferrably endonucleases EcoRI and HindIII, the product of the PCR was ligated into the pcDNA3.1 vector, as known to those skilled in the art.

Mammalian cell culture cells, for example NIH3T3 cells (ATCC, Hanassas, Va., USA) seeded at a density of preferrably 25.000 cells per well in 24-well plates containing Poly-D-Lysine coated coverslips (from BD Biosciences, Erembodegem, Belgium). The day after seeding, cells were transiently transfected with the 2TM-FLAG expression construct described above using Lipofectamin Plus according to the instructions from the manufacturer (for example, InVitroGen, Karlsruhe, Germany). Immunofluorescence on para-formaldehyde fixed cells was performed two days after transfection, as described in the prior art (see, Dorner et. al., J. Biol Chem. (1998) Vol. 273, 20267-75) using a specific antibody against the FLAG-Tag, for example the anti-FLAG M2 antibody (from Sigma, Taufkirchen, Germany), and Cy3-labelled anti-mouse secondary antibody (for example, from Dianova, Hamburg, Germany). In brief, cells were permeabilized with 0.75% triton X-100 for 10 minutes in PBS, endogenous autofluorescence blocked by treatment of the cells with 0.1 NaBH4 in PBS buffer (PBS, 0.5% BSA, 5% goat serum, 0.045% fish gelatine) for 1 hour. Primary antisera (anti-FLAG M2 antibody, at preferrably 0.2 microgram/ml; overnight) and secondary antibody (anti-mouse Cy3-labelled, preferrably 1:400 dilution, 1 hour) were applied in blocking buffer followed by washes in blocking buffer. The cover slips were mounted on glass slides and immunostained cells were examined in an fluorescence microscope with the appropriate filter set for Cy3 at 630× magnification.

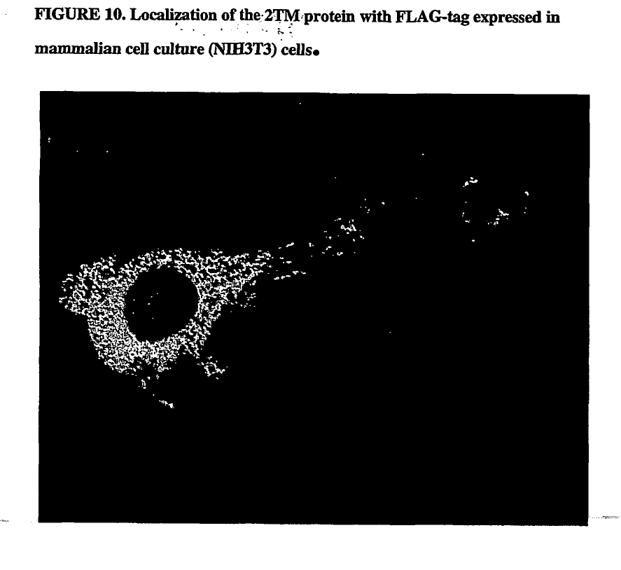

The immunofluorescence as shown in FIG. 10 reveals a clear localization of the mammalian 2TM protein in mitochondria of NIH3T3 cells.

All publications and patents mentioned in the above specification are herein incorporated by reference.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A pharmaceutical composition comprising a nucleic acid molecule of the Unc-51, regulator of mitochondrial activity 1 (ROMA1), and/or mitochondrial 2TM gene family or a polypeptide encoded thereby or a fragment or a variant of said nucleic acid molecule or said polypeptide or an antibody, an aptamer or another receptor recognizing said nucleic acid molecule or polypeptide encoded thereby together with pharmaceutically acceptable carriers, diluents and/or adjuvants.

2. The composition of claim 1, wherein the nucleic acid molecule is a vertebrate or insect Unc-51, ROMA1, and/or 2TM nucleic acid, particularly a human nucleic acid such as human Unc-51-like kinase 1 (ULK-1) (Genbank Accession No. NM 003565) or human KIAA 0623 gene (ULK-2) (Genbank Accession No. NM.01 4683) or human BAP37 (Genbank Accession No. XP 006639.1) or human mitochondrial 2TM (SEQ ID NO:7, SEQ ID NO:6, SEQ ID NO:8, or SEQ ID NO:9) or a mouse nucleic acid such as mouse Unc-51-like kinase 1 (ULK-1) (Genbank Accession No. NM 009469) or mouse Unc-51-like kinase 2 (ULK-2) (Genbank Accession No. AB 019577) or mouse BAP37 (Genbank Accession No. NP 031557.1) or mouse 2TM (Genbank accession number BAB26124), or an insect nucleicacid such as Drosophila melanogaster Unc-51 (GadFly Accession Number CG 10967), ROMA1 (GadFly Accession Number CG15081), and/or 2TM (GadFly Accession Number CG7620), or a fragment thereof or a variant thereof.

3. The composition of claim 1, wherein said nucleic acid molecule
(a) comprises a nucleotide sequence encoding one of the proteins mentioned in claim 2 or the complement thereof;
(b) a nucleotide sequence which hybridizes at 65 or 66° C. in a solution containing 0.2×SSC and 0.1% SDS to the complementary strand of a nucleic acid molecule encoding one of the amino acid sequences of claim 2;
(c) is degenerate with respect to the nucleic acid molecule of (a) and/or (b);
(d) encodes a polypeptide which is at least 85%, preferably at least 90%, more preferably at least 95%, more preferably at least 98% and up to 99.6% identical to one of the proteins of claim 2;
(e) differs from the nucleic acid molecule of (a) to (d) by mutation and wherein said mutation causes an alteration, deletion, duplication or premature stop in the encoded polypeptide or (f comprises a partial sequence of any of the nucleotide sequences of (a) to (e) having a length of at least 15 bases.

4. The composition of claim 1, wherein the nucleic acid molecule is a DNA molecule, particularly a cDNA or a genomic DNA.

5. The composition of claim 1, wherein said nucleic acid encodes a polypeptide contributing to membrane stability and/or function of organelles.

6. The composition of claim 1, wherein said nucleic acid encodes a polypeptide which is a regulator of a transporter molecule.

7. The composition of claim 1, wherein said nucleic acid encodes a polypeptide which is a modifier of mitochondrial proteins.

8. The composition of claim 1, wherein said nucleic acid molecule is a recombinant nucleic acid molecule.

9. The composition of claim 1, wherein said nucleic acid molecule is a DNA or an RNA.

10. The composition of claim 1, wherein the nucleic acid molecule is a vector, particularly an expression vector.

11. The composition of claim 1, wherein the polypeptide is a recombinant polypeptide.

12. The composition of claim 11, wherein said recombinant polypeptide is a fusion polypeptide.

13. The composition of claim 1, wherein said nucleic acid molecule is selected from hybridization probes, primers and 15 anti-sense oligonucleotides.

14. The composition of claim 1 which is a diagnostic composition.

15. The composition of claim 1 which is a therapeutic composition.

16. Use of the composition of claim 1 for the manufacture of an agent for detecting and/or verifying, for the diagnosis, for the treatment, alleviation and/or prevention of a disorder, wherein such disorder is a metabolic disorder or a mitochondrial disorder such as obesity, adipositas, eating/body weight disorders (bulimia nervosa, anorexia nervosa), cachexia (wasting), pancreatic dysfunction(diabetes), mitochondrial
disorders, and/or a disorder related to ROS production and others, in cells, cell masses, organs and/or subjects.

17. Use of a nucleic acid molecule of the Unc-51, ROMA1, and/or 2TM gene family or a polypeptide encoded thereby or a fragment or a variant of said nucleic acid molecule or said polypeptide or an antibody, an aptamer or another receptor recognizing said nucleic acid molecule of or a polypeptide encoded thereby for controlling the function of a gene and/or a gene product which is influenced and/or modified by an Unc-51, ROMA1, and/or 2TM polypeptide.

18. The use of claim 17, wherein said gene and/or gene product is a 10 gene and/or gene product expressed in organelles, wherein said organelle is a mitochondrium, a peroxisome or a chloroplast.

19. Use of a nucleic acid molecule of the Unc-51, ROMA 1, and/or 2TM gene family or a polypeptide encoded thereby or a fragment or a variant of said nucleic acid molecule or said polypeptide or an antibody, an aptamer or another receptor recognizing said nucleic acid molecule or a polypeptide encoded thereby for identifying substances capable of interacting with an Unc-51-kinase-like, ROMA1, and/or 2TM polypeptide.

20. The use of claim 19, wherein said substances capable of interacting with said polypeptide are antagonists or agonists.

21. A non-human transgenic animal exhibiting a modified expression of an Unc-51-kinase-like, ROMA 1, and/or 2TM polypeptide.

22. The animal of claim 21, wherein the expression of the Unc-51 kinase-like, ROMA1, and/or 2TM polypeptide is increased and/or reduced.

23. A recombinant host cell exhibiting a modified expression of an Unc-51-kinase-like, ROMA1, and/or 2TM polypeptide.

24. The cell of claim 23 which is a human cell.

25. A method of identifying a polypeptide or a substance involved in cellular metabolism in an animal or capable of modifying homeostasis comprising the steps of:
(a) testing a collection of polypeptides or substances for interaction with an Unc-51, ROMA1, and/or 2TM polypeptide or a fragment thereof using a readout system; and
(b) identifying polypeptides or substances which test positive for interaction in step (a),
(c) repeating steps (a) and (b) with the polypeptides identified one or more times wherein the newly identified polypeptide replaces the previously identified polypeptide as a bait for the identification of a further interacting polypeptide.

26. The method of claim 25 further comprising the step of identifying the nucleic acid molecule(s) encoding the one or more interacting (poly)peptides.

27. A method of identifying a polypeptide involved in the regulation of body weight in a mammal comprising the steps of
(a) contacting a collection of (poly)peptides with an Unc-51, ROMA1, and/or 2TM like polypeptide or a fragment thereof under conditions that allow binding of said (poly)peptides;
(b) removing (poly)peptides from said collection of (poly)peptides that did not bind to said Unc-51, ROMA 1, and/or 2TM polypeptide in step (a); and
(c) identifying (poly)peptides that bind to said Unc-51, ROMA1, and/or 2TM polypeptide.

28. The method of claim 27 further comprising the step of identifying the nucleic acid molecule(s) encoding the one or more binding (poly)peptides.

29. A method of identifying a compound influencing the expression of a nucleic acid molecule of the Unc-51, ROMA1, and/or 2TM gene family or the activity of an Unc-51, ROMA 1, and/or 2TM polypeptide comprising the steps of
(a) contacting a host carrying an expression vector comprising a nucleic acid molecule of Unc-51, ROMA 1, and/or 2TM or a nucleic acid molecule identified by the method of claim 26 or 28 operatively linked to a readout system with a compound or a collection of compounds;
(b) assaying whether said contacting results in a change of signal intensity provided by said readout system; and, optionally,
(c) identifying a compound within said collection of compounds that induces a change of signal in step (b);
wherein said change in signal intensity correlates with a change of expression of said nucleic acid molecule.

30. A method of assessing the impact of the expression of one or more Unc-51, ROMA1, and/or 2TM polypeptides in a non-human animal comprising the steps of
(a) overexpressing or underexpressing a nucleic acid molecule of the Unc-51, ROMA1, and/or 2TM gene family or a nucleic acid molecule obtainable according to the method of claim 26 in said animal; and
(d) determining whether the weight of said animal has increased, decreased, whether metabolic changes are induced and/or whether the eating behaviour is modified.

31. A method of screening for an agent which modulates the interaction of an Unc-51, ROMA1, and/or 2TM polypeptide with a binding target/agent, comprising the steps of
(a) incubating a mixture comprising
(aa) an Unc-51, ROMA1, and/or 2TM polypeptide, or a fragment thereof or a fusion protein or a fragment thereof;
(ab) a binding target/agent of said (poly)peptide or fusion protein or fragment thereof; and
(ac) a candidate agent
under conditions whereby said (poly)peptide, fusion protein or fragment thereof specifically binds to said binding target/agent at a reference affinity;
(b) detecting the binding affinity of said (poly)peptide, fusion protein or fragment thereof to said binding target to determine an (candidate) agent-biased affinity; and
(c) determining a difference between (candidate) agent-biased affinity and the reference affinity.

32. A method for producing a composition comprising the polypeptide identified by the method of claim 25 with a pharmaceutically acceptable carrier, diluent and/or adjuvant.

33. The method of claim 32 wherein said composition is a pharmaceutical composition for preventing, alleviating or treating obesity, adipositas, eating disorders, wasting syndromes (cachexia), mitochondrial disorders, pancreatic dysfunctions (for example diabetes), disorders related to ROS production.

34. A composition comprising (a) an inhibitor or stimulator of an Unc-51, ROMA1, and/or 2TM (poly)peptide or of a (poly)peptide identified by the method of claim 25.

35. The composition of claim 34 which is a pharmaceutical composition.

36. Use of
(a) an inhibitor or stimulator of the (poly)peptide identified by the method of claim 25;
(b) a modulator of the expression of the gene identified by the method of claim 25;
for the preparation of a pharmaceutical composition for the treatment of obesity, adipositas, eating disorders, wasting syndromes (cachexia), mitochondrial disorders, pancreatic dysfunctions (for example diabetes), disorders related to ROS production.

37. Use of an agent as identified by the method of claim 31 for the preparation of a pharmaceutical composition for the treatment, alleviation and/or prevention of obesity, adipositas, eating disorders, wasting syndromes (cachexia), mitochondrial disorders, pancreatic dysfunctions (for example diabetes), disorders related to ROS production.

38. Use of a nucleic acid molecule of Unc-51, ROMA1, and/or 2TM or fragment thereof for the preparation of a non-human animal which over-or underexpresses the Unc-51, ROMA 1, and/or 2TM gene product.

39. Kit comprising at least one of
(a) an Unc-51, ROMA 1, and/or 2TM nucleic acid molecule, or a fragment thereof,
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody or a fragment or derivative thereof or an antiserum, an aptamer or another receptor against the nucleic acid of (a) or the polypeptide of (d) or (e); and
(g) an anti-sense oligonucleotide, a hybridization probe or a primer for the nucleic acid of (a).

40. A method for producing a composition comprising the polypeptide identified by the method of claim 27 with a pharmaceutically acceptable carrier, diluent and/or adjuvant.

41. A method for producing a composition comprising the polypeptide identified by the method of claim 31 with a pharmaceutically acceptable carrier, diluent and/or adjuvant.

42. A composition comprising
(a) an inhibitor or stimulator of an Unc-51, ROMA1, and/or 2TM (poly)peptide or of a (poly)peptide identified by the method of claim 27.

43. A composition comprising an inhibitor of the expression of a gene identified by the method of claim 26.

44. A composition comprising an inhibitor of the expression of a gene identified by the method of claim 26.

45. A composition comprising a compound identified by the method of claim 29.


SEQUENCE LISTING (28 sequences)

SEQ ID NO: 1 - DNA, 1253 bp, Drosophila melanogaster
  modified_base (5): a, c, g, t, unknown, or other
cgagnaagtgttactatctaaacacatttcaaacaattcttaacaaacaattccaaacatacaattccacttaccacttaccgaccaaattacgagtttacaatggacaaagctgaacgcgactactggcatcttcgatccttggaaatcgaagaggagccgcgatttccgccaacaaacgtcgctgatccactaaccgcacgcaatctgttccagctctacgtcaacaccttcattggagccaatctggccgagtcgtgtgttttcccattggacgtggccaagacccggatgcaggtagatggcgagcaggccaagaagacgggtaaagcgatgccaactttccgtgcaactcttaccaacatgatccgagtggagggattcaagtcgctctacgccggcttctcggcaatggtgacccgaaactttatcttcaactcgttacgtgttgttctctacgacgttttccggcgcccttttctctaccagaacgaacggaacgaggaagtgctcaagatctacatggcgctgggatgcagcttcaccgcaggctgcattgcccaggcactggccaatccctttgacatcgtcaaggtgcgaatgcagacggaaggacgccgccgccagctgggctatgatgtgcgggtgaacagcatggtgcaggccttcgtggacatctaccgccgtggcggactgcccagtatgtggaagggtgtagggcccagctgcatgcgtgcctgcctgatgacgaccggcgatgtgggcagttacgatatcagtaagcgcaccttcaagcgcctgctggacttggaggaaggcctgccactgcgtttcgtgtcttccatgtgcgccggactaacggcatccgtgctcagcacgccggcgaacgtgatcaagtcgcggatgatgaaccagccggtgaacgagagcggcaagaatctgtactacaagaactccctcgactgcattaggaagctggtcagggaggagggtgtcctcacgttgtataagggcctcatgcccacttggtttcgcctgggaccgttctcagtgctcttttggctgtccgtcgagcagctgcgtcagtggaaaggccagagtggattttaggagcaaactatcaatcttactatcgtattttgtatgtcttttaacacgcaataaaaagggtgcaagtcaaaccatctattatacatattataaatataactttaatcccaaaaaaaaaaaaaaaaactcgtgccgaattcgat

SEQ ID NO: 2 - DNA, 1008 bp, Drosophila melanogaster
atggacaaagctgaacgcgactactggcatcttcgatccttggaaatcgaagaggagccgcgatttccgccaacaaacgtcgctgatccactaaccgcacgcaatctgttccagctctacgtcaacaccttcattggagccaatctggccgagtcgtgtgttttcccattggacgtggccaagacccggatgcaggtagatggcgagcaggccaagaagacgggtaaagcgatgccaactttccgtgcaactcttaccaacatgatccgagtggagggattcaagtcgctctacgccggcttctcggcaatggtgacccgaaactttatcttcaactcgttacgtgttgttctctacgacgttttccggcgcccttttctctaccagaacgaacggaacgaggaagtgctcaagatctacatggcgctgggatgcagcttcaccgcaggctgcattgcccaggcactggccaatccctttgacatcgtcaaggtgcgaatgcagacggaaggacgccgccgccagctgggctatgatgtgcgggtgaacagcatggtgcaggccttcgtggacatctaccgccgtggcggactgcccagtatgtggaagggtgtagggcccagctgcatgcgtgcctgcctgatgacgaccggcgatgtgggcagttacgatatcagtaagcgcaccttcaagcgcctgctggacttggaggaaggcctgccactgcgtttcgtgtcttccatgtgcgccggactaacggcatccgtgctcagcacgccggcgaacgtgatcaagtcgcggatgatgaaccagccggtgaacgagagcggcaagaatctgtactacaagaactccctcgactgcattaggaagctggtcagggaggagggtgtcctcacgttgtataagggcctcatgcccacttggtttcgcctgggaccgttctcagtgctcttttggctgtccgtcgagcagctgcgtcagtggaaaggccagagtggattttag

SEQ ID NO: 3 - protein, 335 aa, Drosophila melanogaster
MDKAERDYWHLRSLEIEEEPRFPPTNVADPLTARNLFQLYVNTFIGANLAESCVFPLDVAKTRMQVDGEQAKKTGKAMPTFRATLTNMIRVEGFKSLYAGFSAMVTRNFIFNSLRVVLYDVFRRPFLYQNERNEEVLKIYMALGCSFTAGCIAQALANPFDIVKVRMQTEGRRRQLGYDVRVNSMVQAFVDIYRRGGLPSMWKGVGPSCMRACLMTTGDVGSYDISKRTFKRLLDLEEGLPLRFVSSMCAGLTASVLSTPANVIKSRMMNQPVNESGKNLYYKNSLDCIRKLVREEGVLTLYKGLMPTWFRLGPFSVLFWLSVEQLRQWKGQSGF

SEQ ID NO: 4 - DNA, 900 bp, Drosophila melanogaster
atggcacagagcaaattgaacgatcttgccggcaagctgggcaaaggtggtccgccgggattgggaatcggactgaaggtcctggccgccgtgggagcagccgcctatggagtcagtcagtccctgtacaccgttgagggtggtcaccgcgccatcatcttcagccgtctgggcggcatccagagcgacatttactccgagggactgcacgtgcgcatcccctggttccagtatcccattatctatgacattcgctcgcgtccccgcaaaatatcctcgcccactggctccaaggatctgcagatgatcaacatctcgctgcgtgtgctgtcgcgccccgattccctgaacctgccctatctgcacaagcagctgggcgtggactacgacgagaaggtgctgccctccatttgcaacgaggtgctgaagagcgtgattgccaagttcaatgcctcgcagcttattacccagcgtcagcaggtatccctactcatccgcaaggagctggtcgagcgcgcccgcgacttcaacatcattctggacgatgtgtccctcaccgagctgagcttcggcaaggaatacacggcggccattgaggccaaacaagtggctcagcaggaggcacagcgggctgtcttctttgtggagcgcgccaagcaggagaagcagcagaagattgttcaggctgagggagaagccgaggctgctaaaatgttaggtctggccgtgaagcagaacccagcctacctgaagctgcgcaagctgcgtgccgctcaaagcattgcacgcacgattgccagctcacagaacaaggtctatttgtcggcggatagcttgatgcttaacatccaggactctggcttcgatgacatgaccgagaaggtttacaaaagtaaatag

SEQ ID NO: 5 - protein, 299 aa, Drosophila melanogaster
MAQSKLNDLAGKLGKGGPPGLGIGLKVLAAVGAAAYGVSQSLYTVEGGHRAIIFSRLGGIQSDIYSEGLHVRIPWFQYPIIYDIRSRPRKISSPTGSKDLQMINISLRVLSRPDSLNLPYLHKQLGVDYDEKVLPSICNEVLKSVIAKFNASQLITQRQQVSLLIRKELVERARDFNIILDDVSLTELSFGKEYTAAIEAKQVAQQEAQRAVFFVERAKQEKQQKIVQAEGEAEAAKMLGLAVKQNPAYLKLRKLRAAQSIARTIASSQNKVYLSADSLMLNIQDSGFDDMTEKVYKSK

SEQ ID NO: 6 - protein, 118 aa, Homo sapiens
MAAPPEPGEPEERKSLKLLGFLDVENTPCARHSILYGSLGSVVAGFGHFLFTSRIRRSCDVGVGGFILVTLGCWFHCRYNYAKQRIQERIAREEIKKKILYEGTHLDPERKHNGSSSN

SEQ ID NO: 7 - protein, 111 aa, Homo sapiens
MASTPEPGEPEERKSLKLLGFLDVENTPCARHSILYGSLGSVVAGFGHFLFTSRIRRSCDVGVGGFILVTLGCWFHCRYNYARQRIQERIAREEIKKKILYEGTHLDPERK

SEQ ID NO: 8 - protein, 117 aa, Homo sapiens
MAAAPEPGEPAKRKLFKLLGISGVENIPCARDSILYGSLGSVVAGFGFLLTSRIRKSCDVGVGGFILVTLGCWFHCRYNYAKQRIQERIAREGIKNKILCESTHLDPERKETKGNSS

SEQ ID NO: 9 - protein, 63 aa, Homo sapiens
RIRRSCDVGVGGFILVTLGCWFHWRYNYAKQRIQERIAREGMKTKIYESTHLEPERKETNSNS

SEQ ID NO: 10 - DNA, 24 bp, Artificial Sequence
  Description of Artificial Sequence Synthetic primer
ctaaacaaacaattccaaacatag

SEQ ID NO: 11 - DNA, 24 bp, Artificial Sequence
  Description of Artificial Sequence Synthetic primer
aaaagacatagaaaatacgatagt

SEQ ID NO: 12 - DNA, 21 bp, Artificial Sequence
  Description of Artificial Sequence Synthetic primer
ccatgctgtgcaaatggtaca

SEQ ID NO: 13 - DNA, 19 bp, Artificial Sequence
  Description of Artificial Sequence Synthetic primer
tcggtacacagccctctcg

SEQ ID NO: 14 - DNA, 27 bp, Artificial Sequence
  Description of Artificial Sequence Synthetic probe
tcagctgcccttgatgagatgttccag

SEQ ID NO: 15 - DNA, 20 bp, Artificial Sequence
  Description of Artificial Sequence Synthetic primer
caccaccagagaagttggca

SEQ ID NO: 16 - DNA, 17 bp, Artificial Sequence
  Description of Artificial Sequence Synthetic primer
ggctgtgcttgaccccg

SEQ ID NO: 17 - DNA, 24 bp, Artificial Sequence
  Description of Artificial Sequence Synthetic probe
cttgtccagcttggaggagccagc

SEQ ID NO: 18 - DNA, 28 bp, Artificial Sequence
  Description of Artificial Sequence Synthetic primer
agaaagcttgtgcccatggcggccgccc

SEQ ID NO: 19 - DNA, 56 bp, Artificial Sequence
  Description of Artificial Sequence Synthetic primer
tatcgaattcctacttgtcatcatcgtccttgtagtcgctgctgttgttggtcttc

SEQ ID NO: 20 - protein, 1050 aa, Homo sapiens
MEPGRGGTETVGKFEFSRKDLIGHGAFAVVFKGRHREKHDLEVAVKCINKKNLAKSQTLLGKEIKILKELKHENIVALYDFQEMANSVYLVMEYCNGGDLADYLHAMRTLSEDTIRLFLQQIAGAMRLLHSKGIIHRDLKPQNILLSNPAGRRANPNSIRVKIADFGFARYLQSNMMAATLCGSPMYMAPEVIMSQHYDGKADLWSIGTIVYQCLTGKAPFQASSPQDLRLFYEKNKTLVPTIPRETSAPLRQLLLALLQRNHKDRMDFDEFFHHPFLDASPSVRKSPPVPVPSYPSSGSGSSSSSSSTSHLASPPSLGEMQQLQKTLASPADTAGFLHSSRDSGGSKDSSCDTDDFVMVPAQFPGDLVAEAPSAKPPPDSLMCSGSSLVASAGLESHGRTPSPSPPCSSSPSPSGRAGPFSSSRCGASVPIPVPTQVQNYQRIERNLQSPTQFQTPRSSAIRRSGSTSPLGFARASPSPPAHAEHGGVLARKMSLGGGRPYTPSPQVGTIPERPGWSGTPSPQGAEMRGGRSPRPGSSAPEHSPRTSGLGCRLHSAPNLSDLHVVRPKLPKPPTDPLGAVFSPPQASPPQPSHGLQSCRNLRGSPKLPDFLQRNPLPPILGSPTKAVPSFDFPKTPSSQNLLALLARQGVVMTPPRNRTLPDLSEVGPFHGQPLGPGLRPGEDPKGPFGRSFSTSRLTDLLLKAAFGTQAPDPGSTESLQEKPMEIAPSAGFGGSLHPGARAGGTSSPSPVVFTVGSPPSGSTPPQGPRTRMFSAGPTGSASSSARHLVPGPCSEAPAPELPAPGHGCSFADPIAANLEGAVTFEAPDLPEETLMEQEHTEILRGLRFTLLFVQHVLEIAALKGSASEAAGGPEYQLQESVVADQISLLSREWGFAEQLVLYLKVAELLSSGLQSAIDQIRAGKLCLSSTVKQVVRRLNELYKASVVSCQGLSLRLQRFFLDKQRLLDRIHSITAERLIFSHAVQMVQSAALDEMFQHREGCVPRYHKALLLLEGLQHMLSDQADIENVTKCKLCIERRLSALLTGICA

SEQ ID NO: 21 - protein, 1051 aa, Mus musculus
MEPGRGGVETVGKFEFSRKDLIGHGAFAVVFKGRHREKHDLEVAVKCINKKNLAKSQTLLGKEIKILKELKHENIVALYDFQEMANSVYLVMEYCNGGDLADYLHTMRTLSEDTVRLFLQQIAGAMRLLHSKGIIHRDLKPQNILLSNPGGRRANPSNIRVKIADFGFARYLQSNMMAATLCGSPMYMAPEVIMSQHYDGKADLWSIGTIVYQCLTGKAPFQASSPQDLRLFYEKNKTLVPAIPRETSAPLRQLLLALLQRNHKDRMDFDEFFHHPFLDASTPIKKSPPVPVPSYPSSGSGSSSSSSSASHLASPPSLGEMPQLQKTLTSPADAAGFLQGSRDSGGSSKDSCDTDDFVMVPAQFPGDLVAEAASAKPPPDSLLCSGSSLVASAGLESHGRTPSPSPTCSSSPSPSGRPGPFSSNRYGASVPIPVPTQVHNYQRIEQNLQSPTQQQTARSSAIRRSGSTTPLGFGRASPSPPSHTDGAMLARKLSLGGGRPYTPSPQVGTIPERPSWSRVPSPQGADVRVGRSPRPGSSVPEHSPRTTGLGCRLHSAPNLSDFHVVRPKLPKPPTDPLGATFSPPQTSAPQPCPGLQSCRPLRGSPKLPDFLQRSPLPPILGSPTKAGPSFDFPKTPSSQNLLTLLARQGVVMTPPRNRTLPDLSEASPFHGQQLGSGLRPAEDTRGPFGRSFSTSRITDLLLKAAFGTQASDSGSTDSLQEKPMEIAPSAGFGGTLHPGARGGGASSPAPVVFTVGSPPSGATPPQSTRTRMFSVGSSSSLGSTGSSSARHLVPGACGEAPELSAPGHCCSLADPLAANLEGAVTFEAPDLPEETLMEQEHTETLHSLRFTLAFAQQVLEIAALKGSASEAAGGPEYQLQESVVADQISQLSREWGFAEQLVLYLKVAELLSSGLQTAIDQIRAGKLCLSSTVKQVVRRLNELYKASVVSCQGLSLRLQRFFLDKQRLLDGIHGVTAERLILSHAVQMVQSAALDEMFQHREGCVPRYHKALLLLEGLQHTLTDQADIENIAKCKLCIERRLSALLSGVYA

SEQ ID NO: 22 - protein, 1036 aa, Homo sapiens
MEVVGDFEYSKRDLVGHGAFAVVFRGRHRQKTDWEVAIKSINKKNLSKSQILLGKEIKILKELQHENIVALYDVQELPNSVFLVMEYCNGGDLADYLQAKGTLSEDTIRVFLHQIAAAMRILHSKGIIHRDLKPQNILLSYANRRKSSVSGIRIKIADFGFARYLHSNMMAATLCGSPMYMAPEVIMSQHYDAKADLWSIGTVIYQCLVGKPPFQANSPQDLRMFYEKNRSLMPSIPRETSPYLANLLLGLLQRNQKDRMDFEAFFSHPFLEQGPVKKSCPVPVPMYSGSVSGSSCGSSPSCRFASPPSLPDMQHIQEENLSSPPLGPPNYLQVSKDSASTSSKNSSCDTDDFVLVPHNISSDHSCDMPMGTAGRRASNEFLVCGGQCQPTVSPHSETAPIPVPTQIRNYQRIEQNLTSTASSGTNVHGSPRSAVVRRSNTSPMGFLRPGSCSPVPADTAQTVGRRLSTGSSRPYSPSPLVGTIPEQFSQCCCGHPQGHDSRSRNSSGSPVPQAQSPQSLLSGARLQSAPTLTDIYQNKQKLRKQHSDPVCPSHTGAGYSYSPQPSRPGSLGTSPTKHLGSSPRSSDWFFKTPLPTIIGSPTKTTAPFKIPKTQASSNLLALVTRHGPAEEQSKDGNEPRECAHCLLVQGSERQRAEQQSKAVFGRSVSTGKLSDQQGKTPICRHQGSTDSLNTERPMDIAPAGACGGVLAPPAGTAASSKAVLFTVGSPPHSAAAPTCTHMFLRTRTTSVGPSNSGGSLCAMSGRVCVGSPPGPGFGSSPPGAEAAPSLRYVPYGASPPSLEGLITFEAPELPEETLMEREHTDTLRHLNVMLMFTECVLDLTAMRGGNPELCTSAVSLYQIQESVVVDQISQLSKDWGRVEQLVLYMKAAQLLAASLHLAKAQIKSGKLSPSTAVKQVVKNLNERYKFCITMRKKLTEKLNRFFSDKQRFIDEINSVTAEKLIYNCAVEMVQSAALDEMFQQTEDIVYRYHKAALLLEGLSRILQDPADIENVHKYKCSIERRLSALCHSTATV

SEQ ID NO: 23 - protein, 1037 aa, Mus musculus
MEVVGDFEYCKRDLVGHGAFAVVFRGRHRQKTDWEVAIKSINKKNLSKSQILLGKEIKILKELQHENIVALYDVQELPNSVFLVMEYCNGGDLADYLQAKGTLSEDTIRVFLHQIAAAMRILHSKGIIHRDLKPQNILLSYANRRKSNVSGIRIKIADFGFARYLHSNTMAATLCGSPMYMAPEVIMSQHYDAKADLWSIGTVIYQCLVGKPPFQANSPQDLRMFYEKNRSLMPSIPRETSPYLANLLLGLLQRNQKDRMDFEAFFSHPFLEQVPVKKSCPVPVPVYSGPVPGSSCSSSPSCRFASPPSLPDMQHIQEENLSSPPLGPPNYLQVSKDSASNSSKNSSCDTDDFVLVPHNISSDHSYDMPMGTTARRASNEFFMCGGQCQPTVSPHSETAPIPVPTQVRNYQRIEQNLISTASSGTNPHGSPRSAVVRRSNTSPMGFLRVGSCSPVPGDTVQTGGRRLSTGSSRPYSPSPLVGTIPEQFSQCCCGHPQGHEARSRHSSGSPVPQTQAPQSLLLGARLQSAPTLTDIYQNKQKLRKQHSDPVCPSHAGAGYSYSPQPSRPGSLGTSPTKHTGSSPRNSDWFFKTPLPTIIGSPTKTTAPFKIPKTQASSNLLALVTRHGPAESQSKDGNDPRECSHCLSVQGSERHRSEQQQSKAVFGRSVSTGKLSEQQVKAPLGGHQGSTDSLNTERPMDVAPAGACGVMLALPAGTAASARAVLFTVGSPPHSATAPTCTHMVLRTRTTSVGSSSSGGSLCSASGRVCVGSPPGPGLGSSPPGAEGAPSLRYVPYGASPPSLEGLITFEAPELPEETLMEREHTDTLRHLNMMLMFTECVLDLTAVRGGNPELCTSAVSLYQIQESVVVDQISQLSKDWGRVEQLVLYMKAAQLLAASLHLAKAQVKSGKLSPSMAVKQVVKNLNERYKFCITMCKKLTEKLNRFFSDKQRFIDEINSVTAEKLIYNCAVEMVQSAALDEMFQQTEDIVYRYHKAAVLLEGLSKILQDPTDVENVHKYKCSIERRLSALCCSTATV

SEQ ID NO: 24 - protein, 835 aa, Drosophila melanogaster
MNIVGEYEYSSKDMLGHGAFAVVYKGRHRKKHMPVAIKCITKKGQLKTQNLLGKEIKILKELTELHHENVVALLDCKESQDCVSLVMEYCNGGDLADYLSVKGTLSEDTVRLFLVQLAGAMKALYTKGIVHRDLKPQNILLSHNYGKTLPAPSKITLKIADFGFARFLNEGAMAATLCGSPMYMAPEVIMSLQYDSKADLWSLGTIVYQCLTGKAPFYAQTPNELKSYYEQNANLAPKIPSGVSPDLRDLLLCLLRRNSKDRISYESFFVHRFLQGKKAAVSPVDMPPLGGTPPAKAKSPLQQQLEQELKLVKLAEQQQKEREEQEAQEDENTVSVVANPAICATITNVGVLCDSENNSGSCSSHEDSDDFVLVPKNLPEDQRQGLAQVQAQPASGGQRPQQQQNQSSPPRPSSLPISEPKPVPAPARRQVARPGPLTVATLGGQQIPRSQPISVKQPRPDQRKSSVSSDINSISPPAVQFAIGTPPTRMRSASGGSLSETPPPHAPSTWQVSPGHSQSPLRRSGNSSPVLPSAALTKLPTLGSPTMLVAPGSLGSIGSAGSGSENNNQHHMLGPRAFTLPELGATGGLHSLLDTGAGGGGEPHAFQAPELSEETLMDREHNETLSKLNFVLALTDCIQEVADSRCAPLSTFMVAGSQSAAQAASADAQQIPPHAPEHCKRAERLVLLVRGLQLLSSGMNLASQQLSNGQLKPSSNVKNALLTMNAKYRSMLFESKRLNGSGLLQKANAFNITADKILYDYALDMCQAAALDELLKNTKNCFERYNTAHILLHSLVQKCNHPQDKMMLNKYRDAVEKRLSILQQHGYIYMTDENA

SEQ ID NO: 25 - protein, 299 aa, Homo sapiens
MAQNLKDLAGRLPAGPRGMGTALKLLLGAGAVAYGVRESVFTVEGGHRAIFFNRIGGVQQDTILAEGLHFRIPWFQYPIIYDIRARPRKISSPTGSKDLQMVNISLRVLSRPNAQELPSMYQRLGLDYEERVLPSIVNEVLKSVVAKFNASQLITQRAQVSLLIRRELTERAKDFSLILDDVAITELSFSREYTAAVEAKQVAQQEAQRAQFLVEKAKQEQRQKIVQAEGEAEAAKMLGEALSKNPGYIKLRKIRAAQNISKTIATSQNRIYLTADNLVLNLQDESFTRGSDSLIKGKK

SEQ ID NO: 26 - protein, 298 aa, Mus musculus
MAQNLKDLAGRLPAGPRGMGTALKLLLGAGAVAYGVRESVFTVEGGHRAIFFNRIGGVQQDTILAEFHFRIPWFQYPIIYDIRARPRKISSPTGSKDLQMVNISLRVLSRPNAQELPSMYQRLGLDYEERVLPSIVNEVLKSVVAKFNASQLITQRAQVSLLIRRELTERAKDFSLILDDVAITELSFSREYTAAVEAKQVAQQEAQRAQFLVEKAKQEQRQKIVQAEGEAEAAKMLGEALSKNPGYIKLRKIRAAQNISKTIATSQNRIYLTADNLVLNLQDESFTRGSDSLIKGKK

SEQ ID NO: 27 - protein, 117 aa, Mus musculus
MAAAPEPHETEKKPFKLLGILDVENTPCARESILYGSLGSIVTGLGHFLVTSRIRRSCDVGVGGFILVTLGCWFHCRYNFAKQRIQERIAREGIKNKILYESTHLDPERKMKTNNSS

SEQ ID NO: 28 - protein, 110 aa, Drosophila melanogaster
MAEEPEEPAKSFVIFGRDVAQIPCFRNSFLYGISGGIGIGLLTFLGTSRTHLSTHVGFGSFFCGTIAYWMTCRYQWSVRRFEQQQLREAMRRQALYEGTQRERDLDLKSA